(19) United States
(12) Patent Application Publication
(10) Pub. No.: US 2024/0180844 A1
(43) Pub. Date: Jun. 6, 2024

(54) PARTICLE COMPOSITIONS COMPRISING POLYSARCOSINE LIPID CONJUGATES

(71) Applicant: CALUSA BIO, LLC, Tampa, FL (US)

(72) Inventors: Bradford T. Sullivan, Clearwater, FL (US); Kevin N. Sill, Tampa, FL (US)

(73) Assignee: CALUSA BIO, LLC, Tampa, FL (US)

(21) Appl. No.: 18/388,724

(22) Filed: Nov. 10, 2023

Related U.S. Application Data
(60) Provisional application No. 63/424,617, filed on Nov. 11, 2022.

(51) Int. Cl. A61K 9/51; A61K 48/00

(52) U.S. Cl. CPC A61K 9/5146; A61K 9/5123; A61K 48/0041

(57) ABSTRACT

The present disclosure features particle compositions, such as liposomes and lipid nanoparticles, comprising a polysarcosine-lipid conjugate, as well as methods of making and using the same.

### CLAIM OF PRIORITY

The instant application claims priority to U.S. Application No. 63/424,617, filed on Nov. 11, 2022. The entire contents of the foregoing application are incorporated herein by reference in their entirety.

### BACKGROUND

Biological drug products (biologics) are generally large, complex molecules produced through biotechnology techniques in a living system such as a microorganism, plant cell, or animal cell. Biologics can be more difficult to deliver to a target cell or subject than small molecule drugs. Particle formulations, including lipid nanoparticles, have been used in recent years as a method for delivery of therapeutic payloads; however, these formulations must be fine-tuned across a number of parameters to ensure performance, including stability and specific and efficient payload delivery. Accordingly, it would be desirable to develop particle formulations with improved performance.

### SUMMARY

The present disclosure features particle compositions, such as liposomes and lipid nanoparticles, comprising a polysarcosine-lipid conjugate, as well as methods of making and using the same. In an embodiment, the particle compositions further comprise a therapeutic payload, such an oligonucleotide (e.g., mRNA or DNA) or a protein (e.g., an antibody or enzyme). In an embodiment, the particle compositions comprise a particle comprising: (i) a polymer described herein; and one or more of: (ii) a phospholipid; (iii) a steroid; (iv) a payload (e.g., an oligonucleotide or a protein); and (v) an additional lipid component. In an embodiment, the polymer has the structure of Formula (I-d):

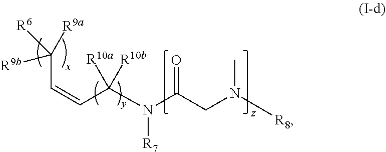

or a pharmaceutically acceptable salt thereof, wherein each of R⁶, R⁷, and R⁸ is independently hydrogen, heteroalkyl, alkyl, alkenyl, or alkynyl; each of R^{9a}, R^{9b}, R^{10a}, and R^{10b} is independently hydrogen, alkyl, or halo; each of x and y is independently an integer between 1 and 20; and z is an integer between 5 and 90. In an embodiment, the particle further comprises (ii). In an embodiment, the particle further comprises (iii). In an embodiment, the particle further comprises (iv). In an embodiment, the particle further comprises (v). In an embodiment, the particle further comprises (vi). Features of the particle compositions comprising a polysarcosine-lipid conjugate, as well as methods of making and methods of use, will be described herein in further detail.

### BRIEF DESCRIPTION OF THE DRAWINGS

FIG. 1 Flow diagram for the synthesis of lipid nanoparticle formulations.

### DETAILED DESCRIPTION

Described herein are particle compositions, such as liposomes and lipid nanoparticles, comprising a polysarcosine-lipid conjugate, as well as methods of making and using the same. In an embodiment, the particle compositions further comprise a therapeutic payload, such an oligonucleotide (e.g., mRNA or DNA) or a protein (e.g., an antibody or enzyme). Features of the particle compositions comprising a polysarcosine-lipid conjugate will be described herein in further detail.

### Definitions

So that the disclosure may be more readily understood, certain technical and scientific terms used herein are specifically defined below. Unless specifically defined elsewhere in this document, all other technical and scientific terms used herein have the meaning commonly understood by one of ordinary skill in the art to which this disclosure belongs.

As used herein, including the appended claims, the singular forms of words such as “a,” “an,” and “the,” include their corresponding plural references unless the context clearly dictates otherwise.

“About”, when used herein to modify a numerically defined parameter (e.g., a physical description of a particle such as diameter, or the amount of a polysarcosine-lipid conjugate in a particle), means that the parameter may vary by as much as 15% above or below the stated numerical value for that parameter. In some embodiments, about means that the parameter may vary by as much as 10% above or below the stated numerical value for that parameter.

“Acquire” or “acquiring”, as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., fluorescence microscope to acquire fluorescence microscopy data.

“Administer”, “administering”, or “administration”, as used herein, refer to providing, absorbing, ingesting, injecting, or otherwise introducing an entity described herein or a composition comprising said particles) or providing the same to a subject.

The term “subject”, as used herein, means a mammal, and includes human and animal subjects, such as domestic animals (e.g., horses, dogs, cats, etc.).

The term “parenteral” or “parenterally” as used herein includes subcutaneous, intravenous, intramuscular, intra-articular, intra-synovial, intrasternal, intrathecal, intrahepatic, intralesional and intracranial injection or infusion techniques for administration. Preferably, the compositions are administered intraperitoneally or intravenously. Sterile injectable forms of the compositions of this disclosure may be aqueous or oleaginous suspension. These suspensions may be formulated according to techniques known in the art using dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution.

As used herein, the term “effective amount” as used herein refers to an amount of a composition of particles (e.g., a particle composition) or a particle component, e.g, a protein or nucleic acid. In some embodiments, the term “effective amount” refers to the amount of a particle component, e.g., the concentration or identity of a therapeutic agent on or within a particle. As will be appreciated by those of ordinary skill in this art, the effective amount may vary depending on such factors as the desired biological endpoint, the pharmacokinetics of the therapeutic agent, composition or particle, the condition being treated, the mode of administration, and the age and health of the subject. An effective amount encompasses therapeutic and prophylactic treatment.

The term “in solution” as used herein, when in reference to a protein, refers to a liquid medium in which the protein is distributed continuously forming a homogenous mixture.

As used herein, the terms “treatment,” “treat,” and “treating” refer to partially or completely alleviating, inhibiting, delaying onset of, slowing the progression of, ameliorating and/or relieving a disease or disorder, or one or more symptoms of the disease or disorder, as described herein. In some embodiments, treatment may be administered after one or more symptoms have developed. In some embodiments, the term “treating” includes preventing, slowing, or halting the progression of a disease or disorder. In some embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder.

### Selected Chemical Definitions

Definitions of specific functional groups and chemical terms are described in more detail below. The chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5th Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987. The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₅-C₆, C₄-C₅, and C₅-C₆. The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₁), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight-chain or branched hydrocarbon group having from 2 to 24 carbon atoms, one or more carbon-carbon double bonds, and no triple bonds (“C2-C24 alkenyl”). In some embodiments, an alkenyl group has 2 to 10 carbon atoms (“C2-C10 alkenyl”). In some embodiments, an alkenyl group has 2 to 8 carbon atoms (“C2-C8 alkenyl”). In some embodiments, an alkenyl group has 2 to 6 carbon atoms (“C2-C6 alkenyl”). In some embodiments, an alkenyl group has 2 carbon atoms (“C2 alkenyl”). The one or more carbon-carbon double bonds can be internal (such as in 2-butenyl) or terminal (such as in 1-butenyl). Examples of C2-C4 alkenyl groups include ethenyl (C2), 1-propenyl (C3), 2-propenyl (C3), 1-butenyl (C4), 2-butenyl (C4), butadienyl (C4), and the like. Examples of C2-C6 alkenyl groups include the aforementioned C2-4 alkenyl groups as well as pentenyl (C5), pentadienyl (C5), hexenyl (C6), and the like. Additional examples of alkenyl include heptenyl (C7), octenyl (C8), octatrienyl (C8), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C1-C10 alkenyl. In certain embodiments, the alkenyl group is substituted C2-C6 alkenyl.

As used herein, the term “alkynyl” refers to a radical of a straight-chain or branched hydrocarbon group having from 2 to 24 carbon atoms, one or more carbon-carbon triple bonds (“C2-C24 alkenyl”). In some embodiments, an alkynyl group has 2 to 10 carbon atoms (“C2-C10 alkynyl”). In some embodiments, an alkynyl group has 2 to 8 carbon atoms (“C2-C8 alkynyl”). In some embodiments, an alkynyl group has 2 to 6 carbon atoms (“C2-C6 alkynyl”). In some embodiments, an alkynyl group has 2 carbon atoms (“C2 alkynyl”). The one or more carbon-carbon triple bonds can be internal (such as in 2-butynyl) or terminal (such as in 1-butynyl). Examples of C2-C4 alkynyl groups include ethynyl (C2), 1-propynyl (C3), 2-propynyl (C3), 1-butynyl (C4), 2-butynyl (C4), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C2-10 alkynyl. In certain embodiments, the alkynyl group is substituted C2-6 alkynyl. “Amino” as used herein refers to the radical —NR70R71, wherein R70 and R71 are each independently hydrogen, C1-C8 alkyl, C3-C10 cycloalkyl, C4-C10 heterocyclyl, C6-C10 aryl, and C5-C10 heteroaryl. In some embodiments, amino refers to NH2.

As used herein, “cyano” refers to the radical —CN.

As used herein, “halo” or “halogen,” independently or as part of another substituent, mean, unless otherwise stated, a fluorine (F), chlorine (Cl), bromine (Br), or iodine (I) atom.

As used herein, “hydroxy” refers to the radical —OH.

As used herein, “oxo” refers to a carbonyl, i.e., —C(O)—.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF3, —CC13, —CH2-CF3, —CH2-CC13, —CH2-CBr3, —CH2-CI3, —CH2-CH2-CH(CF3)-CH3, —CH2-CH2-CH(Br)—CH3, and —CH2-CH═CH—CH2-CF3. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent. As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH2-CH2-O—CH3, —CH2-CH2-NH—CH3, —CH2-CH2-N(CH3)-CH3, —CH2-S—CH2-CH3, —CH2-CH2, —S(O)—CH3, —CH2-CH2-S(O)2-CH3, —CH═CH—O—CH3, —Si(CH3)3, —CH2-CH═N—OCH3, —CH═CH—N(CH3)-CH3, —O—CH3, and —O—CH2-CH3. Up to two or three heteroatoms may be consecutive, such as, for example, —CH2-NH—OCH3 and —CH2-O—Si(CH3)3. Where “heteroalkyl” is recited, followed by recitations of specific heteroalkyl groups, such as —CH2O, —NRCRD, or the like, it will be understood that the terms heteroalkyl and —CH2O or —NRCRD are not redundant or mutually exclusive. Rather, the specific heteroalkyl groups are recited to add clarity. Thus, the term “heteroalkyl” should not be interpreted herein as excluding specific heteroalkyl groups, such as —CH2O, —NRCRD, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C6-C14 aryl”). In some embodiments, an aryl group has six ring carbon atoms (“C6 aryl”; e.g., phenyl). In some embodiments, an aryl group has ten ring carbon atoms (“C10 aryl”; e.g., naphthyl such as 1-naphthyl and 2-naphthyl). In some embodiments, an aryl group has fourteen ring carbon atoms (“C14 aryl”; e.g., anthracyl). An aryl group may be described as, e.g., a C6-C10-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C6-C14 aryl. In certain embodiments, the aryl group is substituted C6-C14 aryl.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent. Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, “cycloalkyl” refers to a radical of a non-aromatic cyclic hydrocarbon group having from 3 to 10 ring carbon atoms (“C3-C10 cycloalkyl”) and zero heteroatoms in the non-aromatic ring system. In some embodiments, a cycloalkyl group has 3 to 8 ring carbon atoms (“C3-C8 cycloalkyl”). In some embodiments, a cycloalkyl group has 3 to 6 ring carbon atoms (“C3-C6 cycloalkyl”). In some embodiments, a cycloalkyl group has 3 to 6 ring carbon atoms (“C3-C6 cycloalkyl”). In some embodiments, a cycloalkyl group has 5 to 10 ring carbon atoms (“C5-C10 cycloalkyl”). A cycloalkyl group may be described as, e.g., a C4-C7-membered cycloalkyl, wherein the term “membered” refers to the non-hydrogen ring atoms within the moiety. Exemplary C3-C6 cycloalkyl groups include, without limitation, cyclopropyl (C3), cyclopropenyl (C3), cyclobutyl (C4), cyclobutenyl (C4), cyclopentyl (C5), cyclopentenyl (C5), cyclohexyl (C6), cyclohexenyl (C6), cyclohexadienyl (C6), and the like. Exemplary C3-C8 cycloalkyl groups include, without limitation, the aforementioned C3-C6 cycloalkyl groups as well as cycloheptyl (C7), cycloheptenyl (C7), cycloheptadienyl (C7), cycloheptatrienyl (C7), cyclooctyl (C8), cyclooctenyl (C8), cubanyl (C8), bicyclo[1.1.1]pentanyl (C5), bicyclo[2.2.2]octanyl (C8), bicyclo[2.1.1]hexanyl (C6), bicyclo[3.1.1]heptanyl (C7), and the like. Exemplary C3-C10 cycloalkyl groups include, without limitation, the aforementioned C3-C8 cycloalkyl groups as well as cyclononyl (C9), cyclononenyl (C9), cyclodecyl (C10), cyclodecenyl (C10), octahydro-1H-indenyl (C9), decahydronaphthalenyl (C10), spiro[4.5]decanyl (C10), and the like.

As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C3-C10 cycloalkyl. In certain embodiments, the cycloalkyl group is a substituted C3-C10 cycloalkyl. “Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl. Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C6 aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

The terms “alkylene,” “alkenylene,” “alkynylene,” “haloalkylene,” “heteroalkylene,” “cycloalkylene,” or “heterocyclylene,” alone or as part of another substituent, mean, unless otherwise stated, a divalent radical derived from an alkyl, alkenyl, alkynyl, haloalkylene, heteroalkylene, cycloalkyl, or heterocyclyl respectively. For example, the term “alkenylene,” by itself or as part of another substituent, means, unless otherwise stated, a divalent radical derived from an alkene. An alkylene, alkenylene, alkynylene, haloalkylene, heteroalkylene, cycloalkylene, or heterocyclylene group may be described as, e.g., a C1-C6-membered alkylene, C2-C6-membered alkenylene, C2-C6-membered alkynylene, C1-C6-membered haloalkylene, C1-C6-membered heteroalkylene, C3-C8-membered cycloalkylene, or C3-C8-membered heterocyclylene, wherein the term “membered” refers to the non-hydrogen atoms within the moiety. In the case of heteroalkylene and heterocyclylene groups, heteroatoms can also occupy either or both of the chain termini (e.g., alkyleneoxy, alkylenedioxy, alkyleneamino, alkylenediamino, and the like).

Still further, no orientation of the linking group is implied by the direction in which the formula of the linking group is written. For example, the formula —C(O)2R′— may represent both —C(O)2R′— and —R′C(O)2-. As used herein, the terms “cyano” or “—CN” refer to a substituent having a carbon atom joined to a nitrogen atom by a triple bond, e.g., C—N. As used herein, the terms “halogen” or “halo” refer to fluorine, chlorine, bromine or iodine. As used herein, the term “hydroxy” refers to —OH. As used herein, the term “nitro” refers to a substitutent having two oxygen atoms bound to a nitrogen atom, e.g., —NO2.

Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure. As used herein, the term “aliphatic” or “aliphatic group”, as used herein, denotes a hydrocarbon moiety that may be straight-chain (i.e., unbranched), branched, or cyclic (including fused, bridging, and spiro-fused polycyclic) and may be completely saturated or may contain one or more units of unsaturation, but which is not aromatic. Unless otherwise specified, aliphatic groups contain 1-20 carbon atoms. In some embodiments, aliphatic groups contain 8-20 carbon atoms. In other embodiments, aliphatic groups contain 12-20 carbon atoms. In still other embodiments, aliphatic groups contain 14-20 carbon atoms, and in yet other embodiments aliphatic groups contain 16-20 carbon atoms. The number of carbon atoms present in the aliphatic groups can also be defined prior to recitation of said aliphatic group. For example, the term (C8-C20)aliphatic refers to an aliphatic group as defined herein comprising from 8 to 20 carbon atoms. It is specifically intended that the disclosure includes each and every individual sub-combination of the members of such range. In particular, the term (C1-C6)aliphatic is intended to include C1 aliphatic (e.g., methyl), C2 aliphatic (e.g., ethyl, ethylene or ethylyne), C3 aliphatic, C4 aliphatic, C5 aliphatic and C6 aliphatic). Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl. Exemplary aliphatic groups include, but are not limited to, C24 aliphatic (e.g., didodecyl), C20 aliphatic (e.g., dodecyl), C18 aliphatic (e.g., oleyl, octadecyl), C16 aliphatic (e.g., hexadecyl, dioctyl), C14 aliphatic (e.g., tetradecyl), C12 aliphatic (e.g., dodecyl, dihexyl), and C10 aliphatic (e.g., decyl).

As used herein, the term “hydrophobic aliphatic group” or “hydrophobic aliphatic” as used herein, denotes a moiety which comprises 6 or more carbon atoms which has an overall hydrophobic characteristic. A hydrophobic aliphatic group may be characterized by traits including, but not limited to, a static water contact angle θ>90°). The number of carbon atoms present in the hydrophobic aliphatic groups can also be defined prior to recitation of said hydrophobic aliphatic group. For example, the term “(C6-C20)hydrophobic aliphatic group” refers to an aliphatic group as defined herein comprising from 6 to 20 carbon atoms. Exemplary hydrophobic aliphatic groups include, but are not limited to, oleyl (i.e., CH₃(CH₂)₇CH═CH(CH₂)₇CH₂—), tetradecyl (i.e., CH₃(CH₂)₁₂CH₂—), hexadecyl (i.e., CH₃(CH₂)₁₄CH₂—), octadecyl (i.e., CH₃(CH₂)₁₆CH₂—), dodecyl (i.e., (CH₃(CH₂)₈CH₂)₂—), and didodecyl (i.e., (CH₃(CH₂)₁₀CH₂)₂—).

Protected hydroxyl groups are well known in the art and include those described in detail in Wuts, P. G. M. Protecting Groups in Organic Synthesis, 5^{th} Ed., New York, John Wiley & Sons, 2014, the entirety of which is incorporated herein by reference. Examples of suitably protected hydroxyl groups further include, but are not limited to, esters, carbonates, sulfonates allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benylbenzoate, 2,4,6-trimethylbenzoate. Examples of carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of silyl ethers include trimethylsilyl, triethylsilyl, t-butyldimethylsilyl, t-butyldiphenylsilyl, triisopropylsilyl ether, and other trialkylsilyl ethers. Examples of alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

Protected amines are well known in the art and include those described in detail in Wuts, P. G. M. Greene's Protective Groups in Organic Synthesis, 5^{th} Ed., New Jersey, J. John Wiley & Sons, 2014. Mono-protected amines further include, but are not limited to, aralkylamines, carbamates, allyl amines, amides, and the like. Examples of mono-protected amino moieties include t-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. Di-protected amines include amines that are substituted with two substituents independently selected from those described above as mono-protected amines, and further include cyclic imides, such as phthalimide, maleimide, succinimide, and the like. Di-protected amines also include pyrroles and the like, 2,2,5,5-tetramethyl-[1,2,5]azadisilolidine and the like, and azide.

Protected aldehydes are well known in the art and include those described in detail in Wuts (2014). Protected aldehydes further include, but are not limited to, acyclic acetals, cyclic acetals, hydrazones, imines, and the like. Examples of such groups include dimethyl acetal, diethyl acetal, diisopropyl acetal, dibenzyl acetal, bis(2-nitrobenzyl) acetal, 1,3-dioxanes, 1,3-dioxolanes, semicarbazones, and derivatives thereof.

Protected carboxylic acids are well known in the art and include those described in detail in Wuts (2014). Protected carboxylic acids further include, but are not limited to, optionally substituted C₁₋₂₀ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional protected carboxylic acids include oxazolines and ortho esters.

Protected thiols are well known in the art and include those described in detail in Wuts (2014). Protected thiols further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

As used herein, the terms “CBP-1”, “oleyl-NH-poly(Sar₁₅)”, “oleylamine-Sar₁₅”, “Oleyl-Sar15”, and “CH₃(CH₂)₇CH═CH(CH₂)₇CH₂NH-poly(sarcosine)₁₅”, all refer to the same compound having the following structure:

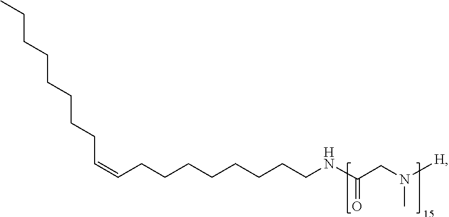

and can be used interchangeably.

As used herein, the terms “CBP-2”, “oleyl-NH-poly(Sar₃₀)”, “oleylamine-Sar₃₀”, “Oleyl-Sar30”, “CH₃(CH₂)₇CH═CH(CH₂)₇CH₂NH-poly(sarcosine)₃₀”, and a polymer having the following structure:

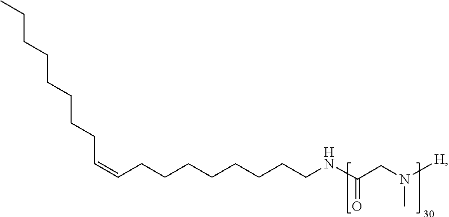

all represent the same compound and can be used interchangeably.

As used herein, terms “CBP-3”, “dodecyl-NH-poly(Sar₂₀)”, “dodecylamine-Sar₂₀”, “Dodecyl-Sar20”, “CH₃(CH₂)₁₀CH₂NH-poly(sarcosine)₂₀”, and a polymer having the following structure:

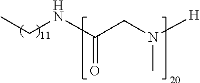

all represent the same compound and can be used interchangeably.

As used herein, the terms “CBP-4”, “tetradecyl-NH-poly(Sar₁₅)”, “tetradecylamine-Sar₁₅”, “Tetradecyl-Sar15”, “CH₃(CH₂)₁₂CH₂NH-poly(sarcosine)₁₅”, and a polymer having the following structure:

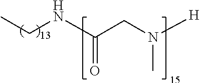

all represent the same compound and can be used interchangeably.

As used herein, terms “CBP-5”, “tetradecyl-NH-poly(Sar₂₀)”, “tetradecylamine-Sar₂₀”, “tetradecyl-Sar20”, “CH₃(CH₂)₁₂CH₂NH-poly(sarcosine)₂₀”, and a polymer having the following structure:

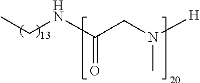

all represent the same compound and can be used interchangeably.

As used herein, the terms “CBP-6”, “hexadecyl-NH-poly(Sar₃₀)”, “hexadecylamine-Sar₃₀”, “Hexadecyl-Sar30”, “CH₃(CH₂)₁₄CH₂NH-poly(sarcosine)₃₀”, and a polymer having the following structure:

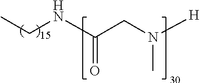

all represent the same compound and can be used interchangeably.

As used herein, the terms “CBP-7”, “octadecyl-NH-poly(Sar₃₀)”, “octadecylamine-Sar₃₀”, Octadecyl-Sar30”, “CH₃(CH₂)₁₆CH₂NH-poly(sarcosine)₃₀”, and a polymer having the following structure:

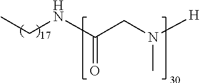

all represent the same compound and can be used interchangeably.

As used herein, the terms “CBP-8”, “didecyl-N-poly(Sar₃₀)”, “didecylamine-Sar₃₀”, “Didecyl-Sar30”, “(CH₃(CH₂)₈CH₂)₂-N-poly(sarcosine)₃₀”, and a polymer having the following structure:

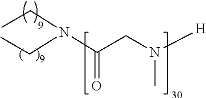

all represent the same compound and can be used interchangeably.

As used herein, the terms “CBP-9”, “didodecyl-N-poly(Sar₃₀), “didodecylamine-Sar₃₀”, “Didodecyl-Sar30”, “(CH₃(CH₂)₁₀CH₂)₂-N-poly(sarcosine)₃₀”, and a polymer having the following structure:

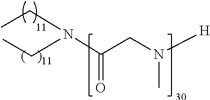

all represent the same compound and can be used interchangeably.

As used herein, the terms “CBP-10”, “oleyl-NH-poly(Sar₁₀)”, “oleylamine-Sar₁₀”, “Oleyl-Sar10”, “CH₃(CH₂)₇CH═CH(CH₂)₇CH₂NH-poly(sarcosine)₁₀”, and a polymer having the following structure:

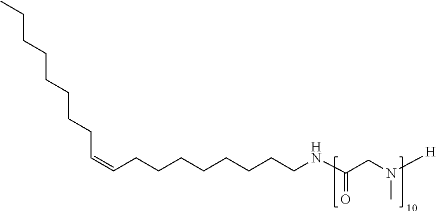

all represent the same compound and can be used interchangeably.

As used herein, the terms “CBP-11”, “tetradecyl-NH-poly(Sar₂₃)”, “tetradecylamine-Sar₂₃”, “tetradecyl-Sar23”, “CH₃(CH₂)₁₂CH₂NH-poly(sarcosine)₂₃”, and a polymer having the following structure:

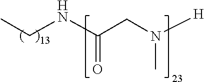

all represent the same compound and can be used interchangeably.

As used herein, the monomer repeat unit described above is a numerical value representing the average number of monomer units comprising the polymer chain. For example, a polymer represented by (A)₁₀ corresponds to a polymer consisting of ten “A” monomer units linked together. One of ordinary skill in the art will recognize that the number 10 in this case will represent a distribution of numbers with an average of 10. The breadth of this distribution is represented by the polydispersity index (PDI). A PDI of 1.0 represents a polymer wherein each chain length is exactly the same (e.g., a protein). A PDI of 2.0 represents a polymer wherein the chain lengths have a Gaussian distribution. Polymers of the present disclosure typically possess a PDI of less than 1.10. In some embodiments, a polymer of the present disclosure has a PDI of about 1.01, about 1.02, about 1.03, about 1.04, about 1.05, about 1.06, about 1.07, about 1.08, about 1.09, about 1.10., about 1.11, about 1.12, about 1.13, about 1.14, about 1.15, about 1.16, about 1.17, about 1.18, about 1.19, or about 1.2.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this disclosure. Such compounds are useful, for example, as in neutron scattering experiments, as analytical tools or probes in biological assays.

As used herein, the term “detectable moiety” is used interchangeably with the term “label” and relates to any moiety capable of being detected (e.g., primary labels and secondary labels). A “detectable moiety” or “label” is the radical of a detectable compound.

“Primary” labels include radioisotope-containing moieties (e.g., moieties that contain ³²P, ³³P, ³⁵S, or ¹⁴C), mass-tags, and fluorescent labels, and are signal-generating reporter groups which can be detected without further modifications.

“Secondary” labels include moieties such as biotin, or protein antigens, that require the presence of a second compound to produce a detectable signal. For example, in the case of a biotin label, the second compound may include streptavidin-enzyme conjugates. In the case of an antigen label, the second compound may include an antibody-enzyme conjugate. Additionally, certain fluorescent groups can act as secondary labels by transferring energy to another compound or group in a process of nonradiative fluorescent resonance energy transfer (FRET), causing the second compound or group to then generate the signal that is detected.

The terms “fluorescent label”, “fluorescent group”, “fluorescent compound”, “fluorescent dye”, and “fluorophore”, as used herein, refer to compounds or moieties that absorb light energy at a defined excitation wavelength and emit light energy at a different wavelength. Examples of fluorescent compounds include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “substrate”, as used herein refers to any material or macromolecular complex to which a polymer can be attached. Examples of commonly used substrates include, but are not limited to, glass surfaces, silica surfaces, plastic surfaces, metal surfaces, surfaces containing a metallic or chemical coating, membranes (e.g., nylon, polysulfone, silica), micro-beads (e.g., latex, polystyrene, or other polymer), porous polymer matrices (e.g., polyacrylamide gel, polysaccharide, polymethacrylate), macromolecular complexes (e.g., protein, polysaccharide).

The term “as received” when referring to the use of a solvent, reagent, resin, or other component used in a chemical reaction or isolation refers to their use in the state provided by the manufacturer without any additional isolation, and/or purification.

As used herein, the terms “protein” or “polypeptide” refer to a polymer of one or more amino acids which are connected via peptide bonds. A protein generally contains greater than 20 such amino acids. The terms include a single polypeptide chain or multiple polypeptide chains complexed together or covalently bound together (e.g., via disulfide bonds).

As used herein, the terms “drug”, “therapeutic agent”, “pharmaceutical”, “medicine” and derivatives thereof, are used interchangeably and refer to a substance intended for use in the diagnosis, cure, mitigation, treatment, or prevention of disease. A drug or therapeutic agent may be a peptide, protein, nucleic acid, small molecule, lipid, cell, or other agent. In an embodiment, the drug or therapeutic agent is encapsulated by the particle described herein.

As used herein, “unit dosage form” or “unit dose form” refers to a physically discrete unit of a formulation appropriate for the subject to be treated. It will be understood, however, that the total daily usage of the compositions of the present disclosure will be decided by the attending physician within the scope of sound medical judgement. The specific effective dose level for any particular subject or organism will depend on a variety of factors including the disorder being treated and the severity of the disorder; activity of specific active agent employed; specific composition employed; age, body weight, general health, sex and diet of the subject; time of administration, and rate of excretion of the specific active agent employed; duration of treatment, drugs/and or additional therapies used in combination or coincidental with specific compound(s) employed and like factors well known in the medical arts.

As used herein, the term “lipid” or “lipid component” refers to a group of organic compounds that include esters of fatty acids and are generally characterized by being poorly soluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids. The lipid or lipid component comprises any lipid known in the art, including a phospholipid, PEGylated lipid, steroid, cationic lipid, anionic lipid, or fatty acid.

As used herein, the term “lipid nanoparticle” refers to particle that comprises a plurality of lipid molecules physically associated with each other by intermolecular forces. Preferably, the lipid nanoparticles are formulated to deliver a payload to one or more target cells. Examples of suitable lipids include, for example, lipids of Formulas (I)-(I-e). The lipid nanoparticles may be, e.g., liposomes/niosomes, nanostructured lipid carriers, microspheres, a dispersed phase in an emulsion, micelles or an internal phase in a suspension.

As used herein, the term “liposome” refers to a closed multilayered structure formed by an outer lipid bilayer enclosing an aqueous inner compartment.

As used herein, the term “phospholipid” refers to any lipid containing a phosphate group or phosphate ion. In some embodiments, “phospholipid” may refer to a triester of glycerol with two aliphatic chains and one alkyl chain containing a phosphate ion. Exemplary phospholipids include naturally occurring phospholipids such as phosphatidylcholine, phosphatidylethanolamine, phosphatidylserine, and phosphatidylglycerol and synthetic phospholipids such as diacylglycerols, phosphatidic acids, phosphocholines, phosphoethanolamines, phosphoglycerols.

As used herein, the term “encapsulate” means to enclose, surround, or encase.

As used herein, “ionizable” refers to a molecule capable of becoming protonated in response to a change in pH. For example, an ionizable lipid is one which may be neutral at physiological pH but is protonated at lower pH values.

### Polysarcosine Lipid Conjugates

The present disclosure relates to polymers comprising a hydrophilic poly(sarcosine) chain and a hydrophobic aliphatic group. In some embodiments, the disclosure provides a polymer of Formula (I):

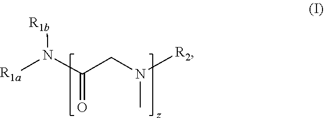

or a pharmaceutically acceptable salt thereof, wherein each of R^{1a}, R^{1b}, and R² is independently hydrogen, alkyl, alkenyl, alkynyl, heteroalkyl, cycloalkyl, heterocyclyl, aryl, or heteroaryl, each of which is optionally substituted with one or more R³; R³ is hydrogen, halo, oxo, cyano, nitro, OR^{C}, alkyl, alkenyl, alkynyl, heteroalkyl, cycloalkyl, heterocyclyl, aryl, or heteroaryl, optionally substituted with one or more R⁴; R^{C} is hydrogen, alkyl, alkenyl, alkynyl, or heteroalkyl; R⁴ is halo, alkyl, heteroalkyl, or OR^{C}; and z is 5-250. In an embodiment, at least one of R^{1a} and R^{1b} is independently alkyl, alkenyl, alkynyl, heteroalkyl, cycloalkyl, heterocyclyl, aryl, or heteroaryl.

In some embodiments, the present disclosure relates to polymers of Formula (I) wherein one or more of R^{1a}, R^{1b}, and R² is alkyl, alkenyl, or alkynyl and the resulting polymer has an overall hydrophobic characteristic. In some embodiments, the present disclosure relates to polymers of Formula (I) wherein R^{1a} is alkyl, alkenyl, or alkynyl and the resulting polymer has an overall hydrophobic characteristic. In some embodiments, the present disclosure relates to polymers of Formula (I) wherein R^{1b} is alkyl, alkenyl, or alkynyl and the resulting polymer has an overall hydrophobic characteristic. In some embodiments, the present disclosure relates to polymers of Formula (I) wherein R² is alkyl, alkenyl, or alkynyl and the resulting polymer has an overall hydrophobic characteristic.

As described above, the present disclosure relates to polymers wherein the hydrophilic chain comprises a polymer of N-methyl glycine (i.e., poly(sarcosine)). The present disclosure further contemplates other N-alkyl glycines which could be used to produce a water-soluble chain (see: Robinson, J. W. et al. Macromolecules 2013, 46(3), 580). In some embodiments, the present disclosure includes polymers wherein the hydrophilic chain is poly(N-methyl glycine), poly(N-ethyl glycine), poly(N-{n-propyl}) glycine, poly(N-isopropyl) glycine, or poly(N-allyl) glycine. In some aspects, the present disclosure also includes mixtures of two or more N-alkyl glycines used to construct the water-soluble chain, such as a mixture of N-methyl glycine and N-ethyl glycine.

Also as described above, in some embodiments one or more of R^{1a}, R^{1b}, and R² in a polymer of Formula (I) are optionally and independently substituted. For instance, in some embodiments, such optional and independent substitutions envisioned by the present disclosure include, but are not limited to, optionally substituted benzyl groups, optionally substituted alkyl, alkenyl, alkynyl, or heteroalkyl groups, optionally substituted silyl groups, poly(amino acid) polymers, poly(ethylene glycol) polymers, poly(N-isopropylacrylamide) polymers, poly(acrylamide) polymers, poly(2-oxazoline) polymers, poly(ethylenimine), poly(acrylic acid) polymers, poly(methacrylate) polymers, poly(vinyl alcohol) polymers, poly(vinylpyrrolidone) polymers, and their corresponding amine salts.

In some embodiments, of Formula (I), R^{1a} is alkyl, alkenyl, alkynyl or cycloalkyl; R^{1b} and R² are hydrogen; and z is 5-30. In some embodiments, of Formula (I), R^{1a} is alkyl; R^{1b} and R² are hydrogen; and z is 5-30. In some embodiments, of Formula (I), R^{1a} is alkenyl; R^{1b} and R² are hydrogen; and z is 5-30. In some embodiments, R^{1a} is alkynyl; R^{1b} and R² are hydrogen; and z is 5-30. In some embodiments, of Formula (I), R^{1a} is C10-C20 alkyl; R^{1b} and R² are hydrogen; and z is 5-30. In some embodiments, of Formula (I), R^{1a} is C10-C20 alkenyl; R^{1b} and R² are hydrogen; and z is 5-30. In some embodiments, R^{1a} is C10-C20 alkynyl; R^{1b} and R² are hydrogen; and z is 5-30.

In some embodiments, of Formula (I), R^{1a} is alkyl optionally substituted with 1 or more R³; R^{1b} and R² are hydrogen; z is 5-30; and R³ is halo, hydroxy, cyano, nitro, oxo or aryl. In some embodiments, of Formula (I), R^{1a} is alkenyl optionally substituted with 1 or more R³; R^{1b} and R² are hydrogen; z is 5-30; and R³ is halo, hydroxy, cyano, nitro, oxo or aryl. In some embodiments, of Formula (I), R^{1a} is alkynyl optionally substituted with 1 or more R³; R^{1b} and R² are hydrogen; z is 5-30; and R³ is halo, hydroxy, cyano, nitro, oxo or aryl.

In some embodiments, of Formula (I), R^{1b} is alkyl, alkenyl, alkynyl or cycloalkyl; R^{1a} and R² are hydrogen; and z is 5-30. In some embodiments, of Formula (I), R^{1b} is alkyl; R^{1a} and R² are hydrogen; and z is 5-30. In some embodiments, of Formula (I), R^{1b} is alkenyl; R^{1a} and R² are hydrogen; and z is 5-30. In some embodiments, R^{1b} is alkynyl; R^{1a} and R² are hydrogen; and z is 5-30. In some embodiments, of Formula (I), R^{1b} is alkenyl optionally substituted with 1 or more R³; R^{1a} and R² are hydrogen; z is 5-30; and R³ is halo, hydroxy, cyano, nitro, oxo or aryl.

In some embodiments, of Formula (I), R^{1a} and R^{1b} are each alkyl; R² is hydrogen; and z is 5-30. In some embodiments, of Formula (I), R^{1a} and R^{1b} are each C5-C11 alkyl; R² is hydrogen; and z is 5-30. In some embodiments, of Formula (I), R^{1a} and R^{1b} are each alkenyl; R² is hydrogen; and z is 5-30. In some embodiments, of Formula (I), R^{1a} and R^{1b} are each C5-C11 alkenyl; R² is hydrogen; and z is 5-30. In some embodiments, of Formula (I), R^{1a} and R^{1b} are each alkynyl; R² is hydrogen; and z is 5-30. In some embodiments, of Formula (I), R^{1a} and R^{1b} are each C5-C11 alkynyl; R² is hydrogen; and z is 5-30.

In some embodiments, of Formula (I), R^{1a} is alkyl; R^{1b} is alkenyl; R² is hydrogen; and z is 5-30. In some embodiments, of Formula (I), R^{1a} is C5-11 alkyl; R^{1b} is C5-C11 alkenyl; R² is hydrogen; and z is 5-30. In some embodiments, of Formula (I), R^{1a} is alkenyl; R^{1b} is alkyl; R² is hydrogen; and z is 5-30. In some embodiments, of Formula (I), R^{1a} is C5-11 alkenyl; R^{1b} is C5-C11 alkyl; R² is hydrogen; and z is 5-30. In some embodiments, of Formula (I), R^{1a} is alkyl; R^{1b} is alkynyl; R² is hydrogen; and z is 5-30. In some embodiments, of Formula (I), R^{1a} is C5-11 alkyl; R^{1b} is C5-C11 alkynyl; R² is hydrogen; and z is 5-30. In some embodiments, of Formula (I), R^{1a} is alkynyl; R^{1b} is alkyl; R² is hydrogen; and z is 5-30. In some embodiments, of Formula (I), R^{1a} is C5-11 alkynyl; R^{1b} is C5-C11 alkyl; R² is hydrogen; and z is 5-30.

In some embodiments, the present disclosure envisions substitutions at R^{1a}, R^{1b}, and R² of a polymer of Formula (I) which may add functionality which would otherwise not be present, including, but not limited to, a detectable moiety, a fluorescent label, or a substrate. Those skilled in the art will recognize that isotopically enriched materials can be useful probes in biological assays, such as quantitative whole-body autoradiography (QWBA) assays useful for determining the distribution of a composition in an animal. In certain embodiments, R^{1a}, R^{1b}, or R² is isotopically enriched. In some embodiments, R^{1a} contains a ¹⁴C isotopically enriched hydrocarbon. In some embodiments, R² contains a ¹⁴C isotopically enriched hydrocarbon.

In some embodiments, the polymer of Formula (I) is Formula (I-a):

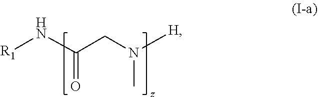

or a pharmaceutically acceptable salt thereof, wherein R¹ is alkyl, alkenyl, alkynyl, or heteroalkyl, each of which is optionally substituted with one or more R³; R³ is hydrogen, halo, oxo, cyano, nitro, OR^{C}, alkyl, alkenyl, alkynyl, heteroalkyl, cycloalkyl, heterocyclyl, aryl, or heteroaryl, optionally substituted with one or more R⁴; R^{C} is hydrogen, alkyl, alkenyl, alkynyl, or heteroalkyl; R⁴ is halo, alkyl, heteroalkyl, or OR^{C}; and z is 5-250.

In some embodiments, of Formula (I-a), R¹ is alkyl; and Z is 10-30. In some embodiments, of Formula (I-a), R¹ is C11-19 alkyl; and z is 10-30. In some embodiments, of Formula (I-a), R¹ is alkenyl; and Z is 10-30. In some embodiments, of Formula (I-a), R¹ is C11-19 alkenyl; and z is 10-30. In some embodiments, of Formula (I-a), R¹ is alkynyl; and Z is 10-30. In some embodiments, of Formula (I-a), R¹ is C11-19 alkynyl; and z is 10-30.

In some embodiments, the polymer of Formula (I) is a polymer of Formula (I-b):

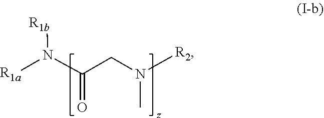

or a pharmaceutically acceptable salt thereof, wherein each of R^{1a} and R^{1b} is (C6-C12)alkyl, (C6-C12)alkenyl, or (C6-C12)alkynyl, each of which is optionally substituted with one or more R³; R³ is hydrogen, halo, oxo, cyano, nitro, OR^{C}, alkyl, alkenyl, alkynyl, heteroalkyl, cycloalkyl, heterocyclyl, aryl, or heteroaryl, optionally substituted with one or more R⁴; R^{C} is hydrogen, alkyl, alkenyl, alkynyl, or heteroalkyl; R⁴ is halo, alkyl, heteroalkyl, or OR^{C} and z is 5-50.

In some embodiments, the disclosure provides a polymer of Formula (I-c):

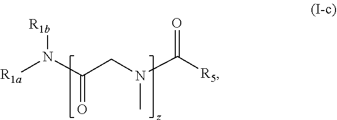

or a pharmaceutically acceptable salt thereof, wherein each of R^{1a} and R^{1b} is hydrogen, (C6-C12)alkyl, (C6-C12)alkenyl, or (C6-C12)alkynyl, each of which is optionally substituted with one or more R³; R⁵ is (C11-C19)alkyl, (C11-C19)alkenyl, or (C11-C19)alkynyl, optionally substituted with one or more R³; R³ is hydrogen, halo, oxo, cyano, nitro, OR^{C}, alkyl, alkenyl, alkynyl, heteroalkyl, cycloalkyl, heterocyclyl, aryl, or heteroaryl, optionally substituted with one or more R⁴; R^{C} is hydrogen, alkyl, alkenyl, alkynyl, or heteroalkyl; R⁴ is halo, alkyl, heteroalkyl, or OR^{C}; and z is 5-50.

In some embodiments, of Formula (I-c), R² is (C11-C18) alkyl. In some embodiments, of Formula (I-c), R² is (C11-C18) alkenyl. In some embodiments, of Formula (I-c), R² is (C11-C18) alkynyl. In some embodiments, of Formula (I-c), R² is C17 alkenyl.

In some embodiments, the disclosure provides a polymer of Formula (I) of Formula (I-d):

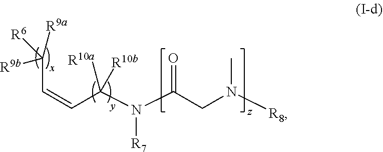

or a pharmaceutically acceptable salt thereof, wherein each of R⁶, R⁷, and R⁸ is independently hydrogen, heteroalkyl, alkyl, alkenyl, or alkynyl; each of R^{9a}, R^{9b}, R^{10a}, and R^{10b} is independently hydrogen, alkyl, or halo; each of x and y is independently an integer between 1 and 20; and z is an integer between 5 and 90.

In some embodiments, of Formula (I-d), R⁶, R⁷, and R⁸ are hydrogen; each of x and y is independently 1-10; and z is 5-30. In some embodiments, of Formula (I-d), R⁶, R⁷, and R⁸ are hydrogen; x is 8; y is 8; and z is 5-30. In some embodiments, of Formula (I-d), R⁶, R⁷, and R⁸ are hydrogen; x is 8; y is 8; and z is 5. In some embodiments, of Formula (I-d), R⁶, R⁷, and R⁸ are hydrogen; x is 8; y is 8; and z is 10. In some embodiments, of Formula (I-d), R⁶, R⁷, and R⁸ are hydrogen; x is 8; y is 8; and z is 15. In some embodiments, of Formula (I-d), R⁶, R⁷, and R⁸ are hydrogen; x is 8; y is 8; and z is 20. In some embodiments, of Formula (I-d), R⁶, R⁷, and R⁸ are hydrogen; x is 8; y is 8; and z is 5-25. In some embodiments, of Formula (I-d), R⁶, R⁷, and R⁸ are hydrogen; x is 8; y is 8; and z is 30.

In some embodiments, the disclosure provides a polymer of Formula (I) of Formula (I-e):

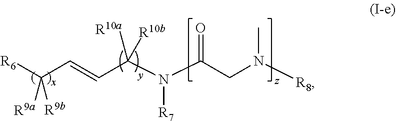

or a pharmaceutically acceptable salt thereof, wherein each of R⁶, R⁷, and R⁸ is independently hydrogen, heteroalkyl, alkyl, alkenyl, or alkynyl; each of R^{9a}, R^{9b}, R^{10a}, and R^{10b} is independently hydrogen, alkyl, or halo; each of x and y is independently an integer between 1 and 20; and z is an integer between 5 and 90.

In some embodiments, of Formula (I-e), R⁶, R⁷, and R⁸ are hydrogen; each of x and y is independently 1-10; and z is 5-30. In some embodiments, of Formula (I-e), R⁶, R⁷, and R⁸ are hydrogen; x is 8; y is 8; and z is 5-30. In some embodiments, of Formula (I-e), R⁶, R⁷, and R⁸ are hydrogen; x is 8; y is 8; and z is 5. In some embodiments, of Formula (I-e), R⁶, R⁷, and R⁸ are hydrogen; x is 8; y is 8; and z is 10. In some embodiments, of Formula (I-e), R⁶, R⁷, and R⁸ are hydrogen; x is 8; y is 8; and z is 15. In some embodiments, of Formula (I-e), R⁶, R⁷, and R⁸ are hydrogen; x is 8; y is 8; and z is 20. In some embodiments, of Formula (I-e), R⁶, R⁷, and R⁸ are hydrogen; x is 8; y is 8; and z is 5-25. In some embodiments, of Formula (I-e), R⁶, R⁷, and R⁸ are hydrogen; x is 8; y is 8; and z is 30.

In some embodiments, the disclosure provides a particle comprising a polymer of Formula (I-f):

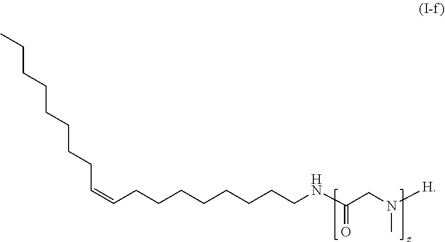

or a pharmaceutically acceptable salt thereof, wherein z is 5-90.

In some such embodiments, of Formula (I-f), z is 10. In some such embodiments, z is 11. In some embodiments, z is 12. In some embodiments, z is 13. In some such embodiments, z is 14. In some such embodiments, z is 15. In some such embodiments, z is 16. In some such embodiments, z is 17. In some such embodiments, z is 18. In some such embodiments, z is 19. In some such embodiments, x is 20. In some such embodiments, z is 21. In some such embodiments, z is 22. In some such embodiments, z is 23. In some such embodiments, z is 24. In some such embodiments, z is 25. In some such embodiments, z is 26. In some such embodiments, z is 27. In some such embodiments, z is 28. In some such embodiments, z is 29. In some such embodiments, x is 30. In some such embodiments, x is 35. In some such embodiments, x is 40. In some such embodiments, x is 45.

In some embodiments, the disclosure provides a particle comprising a polymer of Formula (I-g):

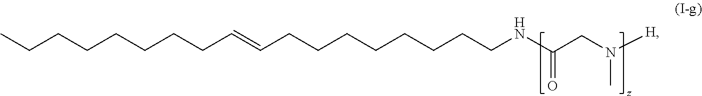

or a pharmaceutically acceptable salt thereof, wherein z is 5-90.

In some such embodiments, of Formula (I-g), z is 10. In some such embodiments, z is 11. In some embodiments, z is 12. In some embodiments, z is 13. In some such embodiments, z is 14. In some such embodiments, z is 15. In some such embodiments, z is 16. In some such embodiments, z is 17. In some such embodiments, z is 18. In some such embodiments, z is 19. In some such embodiments, x is 20. In some such embodiments, z is 21. In some such embodiments, z is 22. In some such embodiments, z is 23. In some such embodiments, z is 24. In some such embodiments, z is 25. In some such embodiments, z is 26. In some such embodiments, z is 27. In some such embodiments, z is 28. In some such embodiments, z is 29. In some such embodiments, x is 30. In some such embodiments, x is 35. In some such embodiments, x is 40. In some such embodiments, x is 45.

In some embodiments, the present disclosure provides a particle comprising a polymer of any of the following structures for use in accord with the present invention:

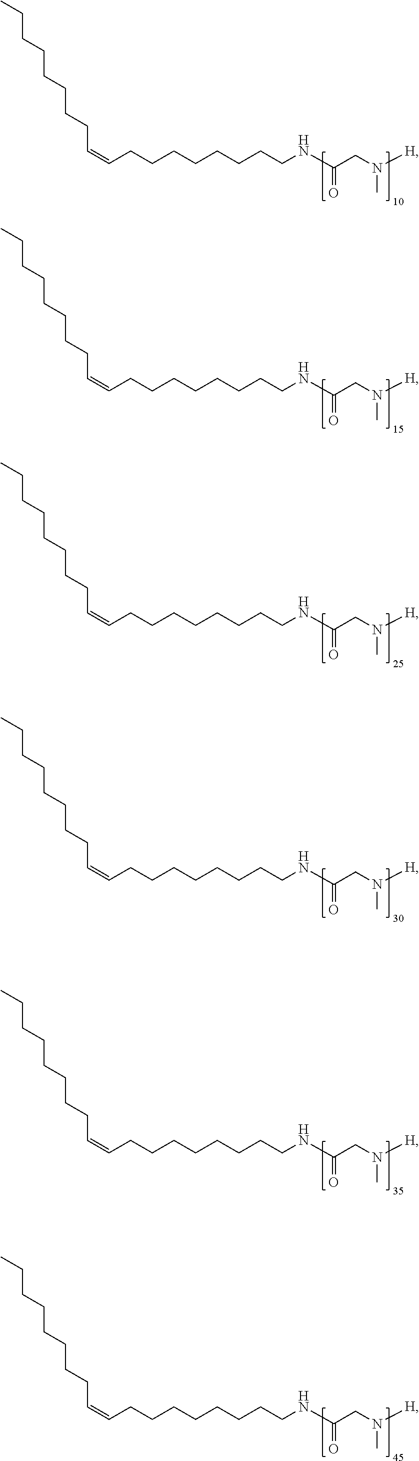

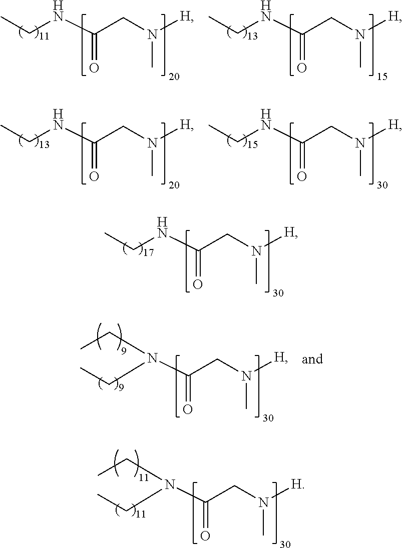

In some embodiments, the present disclosure provides a polymer of the following structure:

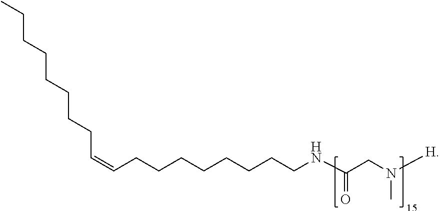

In some embodiments, the present disclosure provides a polymer of the following structure:

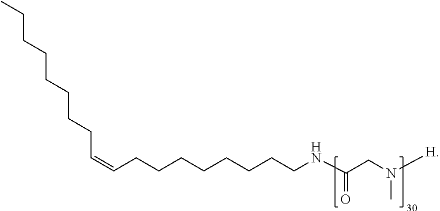

In some embodiments, the present disclosure provides a polymer of the following structure:

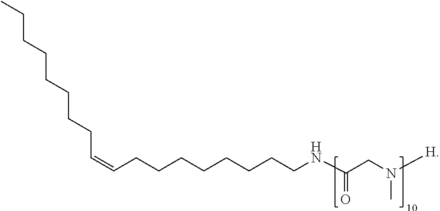

In some embodiments, the present disclosure provides a polymer of the following structure:

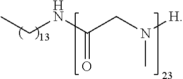

### Lipids and Lipid Components

The present disclosure provides particle compositions (e.g., lipid nanoparticles e.g., liposomes) that comprise a lipid or lipid component, such as a phospholipid, PEGylated lipid, or steroid. In an embodiment, the lipid or lipid component is a phospholipid. In some embodiments, the phospholipid is one of phosphatidylcholine, phosphatidylethanolamine, phosphatidylserine, and phosphatidylglycerol, or derivatives thereof. In some embodiments, the phospholipid is a compound of Formula (II):

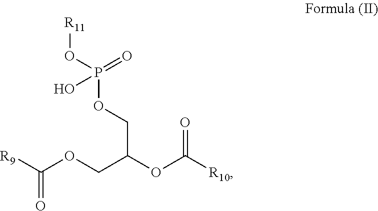

or a pharmaceutically acceptable salt thereof, wherein each R⁹ and R¹⁰ is independently alkyl, alkenyl, or alkynyl, optionally substituted with one or more R³; R¹¹ is alkyl, alkenyl, alkynyl, or heteroalkyl, optionally substituted with one or more R¹²; R³ is hydrogen, halo, oxo, cyano, nitro, OR^{C}, alkyl, alkenyl, alkynyl, heteroalkyl, cycloalkyl, heterocyclyl, aryl, or heteroaryl, optionally substituted with one or more R⁴; R^{C} is hydrogen, alkyl, alkenyl, alkynyl, or heteroalkyl; R⁴ is halo, alkyl, heteroalkyl, or OR^{C}; R¹² is alkyl, alkenyl, alkynyl, —NR^{D}R^{E}, oxo, or hydroxy; and each of R^{D} and R^{E} is independently hydrogen or alkyl.

In some embodiments, the phospholipid is a phosphatidylcholine. In some embodiments, the phospholipid of Formula (II) is a compound of Formula (II-a):

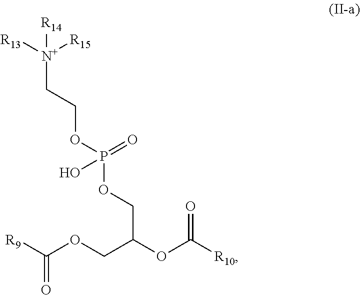

or a pharmaceutically acceptable salt thereof, wherein each R⁹ and R¹⁰ is independently (C1-C25) alkyl, (C2-C25) alkenyl, or (C2-C25) alkynyl, optionally substituted with one or more R³; each of R¹³, R¹⁴, and R¹⁵ is independently alkyl, alkenyl, alkynyl, or heteroalkyl, optionally substituted with one or more R¹²; R³ is hydrogen, halo, oxo, cyano, nitro, OR^{C}, alkyl, alkenyl, alkynyl, heteroalkyl, cycloalkyl, heterocyclyl, aryl, or heteroaryl, optionally substituted with one or more R⁴; R^{C} is hydrogen, alkyl, alkenyl, alkynyl, or heteroalkyl; R⁴ is halo, alkyl, heteroalkyl, or OR^{C}; and R¹² is alkyl, alkenyl, alkynyl, oxo, or hydroxy.

In some embodiments, of Formula (II-a), each of R⁹, R¹⁰, R¹³, R¹⁴, and R¹⁵ is alkyl. In some embodiments, of Formula (II-a), each of R⁹ and R¹⁰ is alkenyl and R¹³, R¹⁴, and R¹⁵ are alkyl. In some embodiments, of Formula (II-a), each of R⁹ and R¹⁰ is C6 alkyl; and each of R¹³, R¹⁴, and R¹⁵ is C1 alkyl. In some embodiments, of Formula (II-a), each of R⁹ and R¹⁰ is C7 alkyl; and each of R¹³, R¹⁴, and R¹⁵ is C1 alkyl. In some embodiments, of Formula (II-a), each of R⁹ and R¹⁰ is C8 alkyl; and each of R¹³, R¹⁴, and R¹⁵ is C1 alkyl.

In some embodiments, the phospholipid is a phosphatidylserine. In some embodiments, the phospholipid of Formula (II) is compound of Formula (II-b):

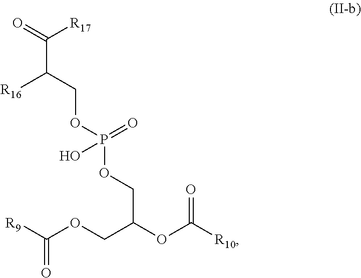

or a pharmaceutically acceptable salt thereof, wherein each R⁹ and R¹⁰ is independently (C1-C25) alkyl, (C2-C25) alkenyl, or (C2-C25) alkynyl, optionally substituted with one or more R³; each of R¹⁶ and R¹⁷ is independently alkyl, alkenyl, alkynyl, or heteroalkyl, optionally substituted with one or more R¹²; R³ is hydrogen, halo, oxo, cyano, nitro, OR^{C}, alkyl, alkenyl, alkynyl, heteroalkyl, cycloalkyl, heterocyclyl, aryl, or heteroaryl, optionally substituted with one or more R⁴; R^{C} is hydrogen, alkyl, alkenyl, alkynyl, or heteroalkyl; R⁴ is halo, alkyl, heteroalkyl, or OR^{C}; R¹² is alkyl, alkenyl, alkynyl, —NR^{D}R^{E}, oxo, or hydroxy; and each of R^{D} and R^{E} is independently hydrogen or alkyl.

In some embodiments, of Formula (II-b), each of R⁹ and R¹⁰ is alkyl; R¹⁶ is —NR^{D}R^{E}; and R¹⁷ is hydroxy; and R^{D} and R^{E} are each hydrogen.

In some embodiments, the phospholipid is a phosphatidylglycerol. In some embodiments, the phospholipid of Formula (II) is a compound of Formula (II-c):

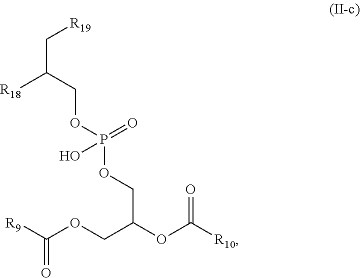

or a pharmaceutically acceptable salt thereof, wherein each R⁹ and R¹⁰ is independently (C1-C25) alkyl, (C2-C25) alkenyl, or (C2-C25) alkynyl, optionally substituted with one or more R³; each of R¹⁸ and R¹⁹ is independently alkyl, alkenyl, alkynyl, or heteroalkyl, optionally substituted with one or more R¹²; R³ is hydrogen, halo, oxo, cyano, nitro, OR^{C}, alkyl, alkenyl, alkynyl, heteroalkyl, cycloalkyl, heterocyclyl, aryl, or heteroaryl, optionally substituted with one or more R⁴; R^{C} is hydrogen, alkyl, alkenyl, alkynyl, or heteroalkyl; R⁴ is halo, alkyl, heteroalkyl, or OR^{C}; R¹² is alkyl, alkenyl, alkynyl, —NR^{D}R^{E}, oxo, or hydroxy; and each of R^{D} and R^{E} is independently hydrogen or alkyl.

In some embodiments, the phospholipid is a phosphatidylethanolamine. In some embodiments, the phospholipid is a compound of Formula (II-d):

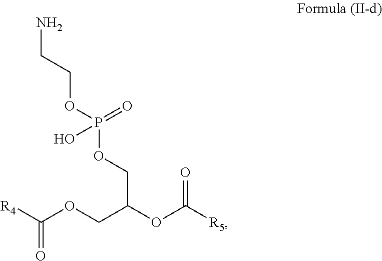

or a pharmaceutically acceptable salt thereof, wherein each R⁹ and R¹⁰ is independently (C1-C25) alkyl, (C2-C25) alkenyl, or (C2-C25) alkynyl, optionally substituted with one or more R³; R³ is hydrogen, halo, oxo, cyano, nitro, OR^{C}, alkyl, alkenyl, alkynyl, heteroalkyl, cycloalkyl, heterocyclyl, aryl, or heteroaryl, optionally substituted with one or more R⁴; R^{C} is hydrogen, alkyl, alkenyl, alkynyl, or heteroalkyl; R⁴ is halo, alkyl, heteroalkyl, or OR^{C}; R¹² is alkyl, alkenyl, alkynyl, —NR^{D}R^{E}, oxo, or hydroxy; and each of R^{D} and R^{E} is independently hydrogen or alkyl.

In some embodiments, the phospholipid is 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, DOPE, 1,2-dioleoyl-sn-glycero-3-phosphoethanolamine; 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), dipalmitoylphosphatidylglycerol (DPPG), palmitoyloleoylphosphatidylethanolamine (POPE), distearoyl-phosphatidyl-ethanolamine (DSPE), dipalmitoyl phosphatidyl ethanolamine (DPPE), dimyristoylphosphoethanolamine (DMPE), 1-stearoyl-2-oleoyl-phosphatidyethanolamine (SOPE), 1-stearoyl-2-oleoyl-phosphatidylcholine (SOPC), sphingomyelin, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine (LPE), decyl (2-(dioctylammonio)ethyl) phosphate; ePC, ethylphosphatidylcholine, or a salt thereof.

In some embodiments, the lipid is a polyethylene glycol modified (PEGylated) lipid or a derivative thereof. In an embodiment, the PEGylated lipid is methoxypolyethylene-glycoloxy(2000)-N,N-ditetradecylacetamide (ALC-0159), 1,2-dimyristoyl-rac-glycero-3-methoxypolyethylene glycol-2000 (DMG-PEG 2000), 1,2-dimyristoyl-rac-glycero-3-methoxypolyethylene glycol-5000 (DMG-PEG 5000), 1,2-dioleoyl-sn-glycero-3-phosphoethanolamine-N-[amino(polyethylene glycol)-2000] (DOPE-PEG(2000), 1,2-dioleoyl-sn-glycero-3-phosphoethanolamine-N-[amino(polyethylene glycol)-5000] (DOPE-PEG(5000), 1,2-distearoyl-sn-glycero-3-phosphoethanolamine-N-[amino(polyethylene glycol)-2000] (DSPE-PEG(2000), 1,2-distearoyl-sn-glycero-3-phosphoethanolamine-N-[amino(polyethylene glycol)-5000] (DSPE-PEG(5000), distearoyl-rac-glycerol-PEG2K (DSG-PEG 2000), distearoyl-rac-glycerol-PEG5K (DSG-PEG 5000).

In some embodiments, the phospholipid is a combination of one or more phospholipids described herein in Formulas (II)-(II-d).

In another aspect, the present disclosure provides particle compositions (e.g., lipid nanoparticles e.g., liposomes) that comprise a steroid. In some embodiments, the steroid is cholesterol or a derivative thereof. In some embodiments, the steroid is a compound of Formula (III):

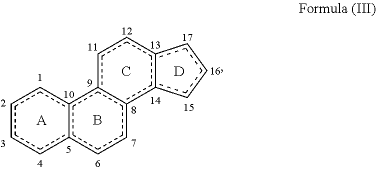

or a pharmaceutically acceptable salt, tautomer, or stereoisomer thereof, wherein each of ring positions 1-17 is optionally substituted with one or more R³; R³ is hydrogen, halo, oxo, cyano, nitro, OR^{C}, alkyl, alkenyl, alkynyl, heteroalkyl, cycloalkyl, heterocyclyl, aryl, or heteroaryl, optionally substituted with one or more R⁴; R^{C} is hydrogen, alkyl, alkenyl, alkynyl, or heteroalkyl; R⁴ is halo, alkyl, alkenyl, alkynyl, heteroalkyl, or OR^{C}; R¹² is alkyl, alkenyl, alkynyl, —NR^{D}R^{E}, oxo, or hydroxy; each of R^{D} and R^{E} is independently hydrogen or alkyl; and dotted lines, “---” represent single or double bonds as determined by rules of chemical valency.

In some embodiments, of Formula (III), ring positions 3, 10, 13, and 17 are each substituted with 1 R³; and the bond between ring positions 5 and 6 is a double bond. In some embodiments, the compound of Formula (III) is gonane or a derivative thereof. In some embodiments, the compound of Formula (III) is sterol or a derivative thereof. In some embodiments, the compound of Formula (III) is cholesterol or a derivative thereof.

In some embodiments, the steroid of Formula (III) is a compound of Formula (III-a):

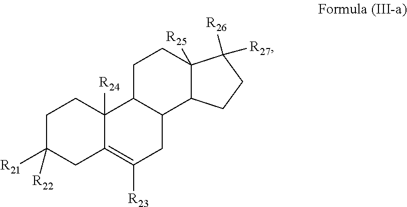

or a pharmaceutically acceptable salt, tautomer, or stereoisomer thereof, wherein each of R²¹, R²², R²³, R²⁴, R²⁵, R²⁶, and R²⁷ is independently hydrogen, oxo, hydroxy, halo, alkyl, alkenyl, alkynyl, or heteroalkyl, optionally substituted with one or more R³; R³ is hydrogen, halo, oxo, cyano, nitro, OR^{C}, alkyl, alkenyl, alkynyl, heteroalkyl, cycloalkyl, heterocyclyl, aryl, or heteroaryl, optionally substituted with one or more R⁴; R^{C} is hydrogen, alkyl, alkenyl, alkynyl, or heteroalkyl; R⁴ is halo, alkyl, alkenyl, alkynyl, heteroalkyl, or OR^{C}; and R¹² is alkyl, alkenyl, alkynyl, —NR^{D}R^{E}, oxo, or hydroxy; each of R^{D} and R^{E} is independently hydrogen or alkyl.

In some embodiments, of Formula (III-a), R²¹ is OR^{C}; R²⁴ and R²⁵ are alkyl (e.g., methyl); R²⁶ is alkyl (e.g., heptyl) substituted with two R³; R²², R²³, and R²⁷ are hydrogen; and R³ is alkyl (e.g., methyl). In some embodiments, of Formula (III-a) the compound of Formula (III-a) is gonane or a derivative thereof. In some embodiments, the compound of Formula (III-a) is a sterol or a derivative thereof. In some embodiments, the compound of Formula (III-a) is cholesterol or a derivative thereof.

In some embodiments, the steroid of Formula (III) is a compound of Formula (III-b):

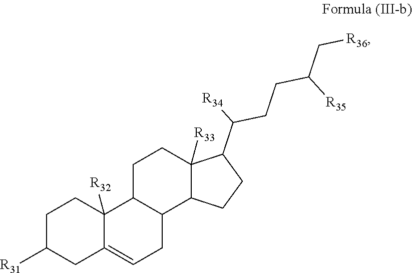

or a pharmaceutically acceptable salt, tautomer, or stereoisomer thereof, wherein each of R³¹, R³², R³³, R³⁴, R³⁵, and R³⁶ is independently hydrogen, oxo, hydroxy, halo, alkyl, alkenyl, alkynyl, or heteroalkyl, optionally substituted with one or more R³; R³ is hydrogen, halo, oxo, cyano, nitro, OR^{C}, alkyl, alkenyl, alkynyl, heteroalkyl, cycloalkyl, heterocyclyl, aryl, or heteroaryl, optionally substituted with one or more R⁴; R^{C} is hydrogen, alkyl, alkenyl, alkynyl, or heteroalkyl; R⁴ is halo, alkyl, alkenyl, alkynyl, heteroalkyl, or OR^{C}; and R¹² is alkyl, alkenyl, alkynyl, —NR^{D}R^{E}, oxo, or hydroxy; each of R^{D} and R^{E} is independently hydrogen or alkyl.

In some embodiments of compound (III-b), R³¹ is OR^{A} (e.g., hydroxyl, e.g., —OH), and R³², R³³, R³⁴, R³⁵, and R³⁶ are alkyl (e.g., methyl).

In some embodiments, the steroid is cholesterol or a cholesterol-derivative. In some embodiments, the cholesterol derivative is Cholesta-5,7-dien-3beta-ol, Cholest-5-en-3-ol, 7beta-Hydroxycholesterol, Cholest-7-en-3beta-ol, Lanosta-8,24-dien-3-ol, (3S,8S,9S,10R,13R,14S,17R)-17-[(2S,5S)-5-Ethyl-6-methyl-heptan-2-yl]-10,13-dimethyl-2,3,4,7,8,9,11,12,14,15,16,17-dodecahydro-1H-cyclopenta[a]phenanthren-3-ol, (3S)-17-[(5S)-5-ethyl-6-methylheptan-2-yl]-10,13-dimethyl-2,3,4,7,8,9,11,12,14,15,16,17-dodecahydro-1H-cyclopenta[a]phenanthren-3-ol, (3S,8S,9S,10R,13R,14S,17R)-17-[(2R,5S)-5-ethyl-6-methylhept-3-en-2-yl]-10,13-dimethyl-2,3,4,7,8,9,11,12,14,15,16,17-dodecahydro-1H-cyclopenta[a]phenanthren-3-ol, 17-[(5R)-5,6-dimethylheptan-2-yl]-10,13-dimethyl-2,3,4,7,8,9,11,12,14,15,16,17-dodecahydro-1H-cyclopenta[a]phenanthren-3-ol, Cholesteryl isopropyl ether, Stigmast-5-en-3-ol, 10-(Iodomethyl)-17-(6-methylheptan-2-yl)-1,2,3,4,7,8,9,11,12,13,14,15,16,17-tetradecahydrocyclopenta[a]phenanthren-3-ol, (3S,5S,10S,13R,17R)-3-hydroxy-10,13-dimethyl-17-(6-methylheptan-2-yl)-1,2,3,4,5,6,7,9,11,12,16,17-dodecahydrocyclopenta[a]phenanthren-15-one, 25-Hydroxycholesterol, Lathosterol, 3-Methoxycholest-5-ene, 17-(5-Ethyl-6-methylhept-6-en-2-yl)-4,10,13-trimethyl-2,3,4,5,6,9,11,12,14,15,16,17-dodecahydro-1H-cyclopenta(a)phenanthren-3-ol, 17-(5,6-dimethylheptan-2-yl)-10,13-dimethyl-2,3,4,5,6,9,11,12,14,15,16,17-dodecahydro-1H-cyclopenta[a]phenanthren-3-ol, (7R,8S,9S,10R,13R,14S,17R)-10,13-dimethyl-17-[(2R)-6-methylheptan-2-yl]-2,3,4,7,8,9,11,12,14,15,16,17-dodecahydro-1H-cyclopenta[a]phenanthrene-3,7-diol, 24-Methylenecholesterol, Zymosterol, BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl)oxy)carbonyl)amino)-N,N-bis(2-hydroxyethyl)-N-methylethan-1-aminium bromide; DC-Cholesterol, 3β-[N—(N′,N′-dimethylaminoethane)-carbamoyl]cholesterol; dihydrocholesterol, ent-cholesterol, epi-cholesterol, desmosterol, cholestanol, cholestanone, cholestenone, cholesteryl-2′-hydroxyethyl ether, cholesteryl-4′-hydroxybutyl ether, 3β-[N—(N′N′-dimethylaminoethyl)carbamoyl cholesterol (DC-Chol), 24(S)-hydroxycholesterol, 25-hydroxycholesterol, 25(R)-27-hydroxycholesterol, 22-oxacholesterol, 23-oxacholesterol, 24-oxacholesterol, cycloartenol, 22-ketosterol, 20-hydroxysterol, 7-hydroxycholesterol, 19-hydroxycholesterol, 22-hydroxycholesterol, 25-hydroxycholesterol, 7-dehydrocholesterol, 5a-cholest-7-en-3β-ol, 3,6,9-trioxaoctan-1-ol-cholesteryl-3e-ol, dehydroergosterol, dehydroepiandrosterone, lanosterol, dihydrolanosterol, lanostenol, lumisterol, sitocalciferol, calcipotriol, coprostanol, cholecalciferol, lupeol, ergocalciferol, 22-dihydroegocalciferol, ergosterol, brassicasterol, tomatidine, tomatine, ursolic acid, cholic acid, chenodeoxycholic acid, zymosterol, diosgenin, fucosterol, fecosterol, or fecosterol, or a salt, stereoisomer, or tautomer thereof.

In some embodiments, the lipid is a cationic lipid or an ionizable lipid (i.e., a lipid capable of being protonated at low pH). In some embodiments, the lipid (e.g., cationic lipid or ionizable lipid) is a compound of Formula (IV):

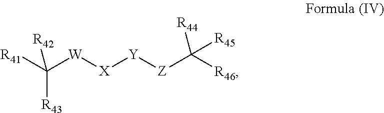

or a pharmaceutically acceptable salt, tautomer, or stereoisomer thereof, wherein each of R⁴¹, R⁴², R⁴³, R⁴⁴, R⁴⁵, and R⁴⁶ is independently hydrogen, oxo, hydroxy, halo, alkyl, alkenyl, alkynyl, or heteroalkyl, optionally substituted with one or more R³; each of W, X, Y, and Z is independently —N(R³)—, —N(R³)(R3′)— or C(R³)(R^{3′})—R³ and R^{3′} are hydrogen, halo, oxo, cyano, nitro, OR^{C}, alkyl, alkenyl, alkynyl, heteroalkyl, cycloalkyl, heterocyclyl, aryl, or heteroaryl, optionally substituted with one or more R⁴; R^{C} is hydrogen, alkyl, alkenyl, alkynyl, or heteroalkyl; R⁴ is halo, alkyl, alkenyl, alkynyl, heteroalkyl, or OR^{C}; and R¹² is alkyl, alkenyl, alkynyl, —NR^{D}R^{E}, oxo, or hydroxy; each of R^{D} and R^{E} is independently hydrogen or alkyl.

In some embodiments, the lipid is a compound of Formula (IV-a):

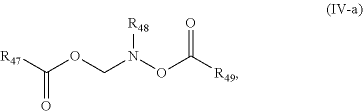

or a pharmaceutically acceptable salt, tautomer, or stereoisomer thereof, wherein each R⁴⁷, R⁴⁸, and R⁴⁹ is independently hydrogen, oxo, hydroxy, halo, alkyl, alkenyl, alkynyl, or heteroalkyl, optionally substituted with one or more R³; R³ is hydrogen, halo, oxo, cyano, nitro, OR^{C}, alkyl, alkenyl, alkynyl, heteroalkyl, cycloalkyl, heterocyclyl, aryl, or heteroaryl, optionally substituted with one or more R⁴; R^{C} is hydrogen, alkyl, alkenyl, alkynyl, or heteroalkyl; R⁴ is halo, alkyl, alkenyl, alkynyl, heteroalkyl, or OR^{C}; and R¹² is alkyl, alkenyl, alkynyl, —NR^{D}R^{E}, oxo, or hydroxy; each of R^{D} and R^{E} is independently hydrogen or alkyl.

In some embodiments, the lipid is a compound of formula (II-b):

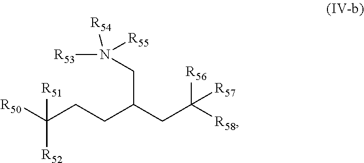

or a pharmaceutically acceptable salt, tautomer, or stereoisomer thereof, wherein R⁵⁰, R⁵¹, R⁵², R⁵³, R⁵⁴, R⁵⁵, R⁵⁶, R⁵⁷, and R⁵⁸ is independently hydrogen, oxo, hydroxy, halo, alkyl, alkenyl, alkynyl, or heteroalkyl, optionally substituted with one or more R³; R³ is hydrogen, halo, oxo, cyano, nitro, OR^{C}, alkyl, alkenyl, alkynyl, heteroalkyl, cycloalkyl, heterocyclyl, aryl, or heteroaryl, optionally substituted with one or more R⁴; R^{C} is hydrogen, alkyl, alkenyl, alkynyl, or heteroalkyl; R⁴ is halo, alkyl, alkenyl, alkynyl, heteroalkyl, or OR^{C}; and R¹² is alkyl, alkenyl, alkynyl, —NR^{D}R^{E}, oxo, or hydroxy; each of R^{D} and R^{E} is independently hydrogen or alkyl.

In some embodiments, the 1 lipid (e.g., cationic lipid or ionizable lipid) is selected from a lipid described in International Publication Nos. WO2012040184, WO2011153120, WO2011149733, WO2011090965, WO2011043913, WO2011022460, WO2012061259, WO2012054365, WO2012044638, WO2010080724, WO201021865 and WO2008103276, U.S. Pat. Nos. 7,893,302, 7,404,969 and 8,283,333 and US Patent Publication No. US20100036115 and US20120202871.

In some embodiments, the lipid (e.g., any lipid, cationic lipid, or ionizable lipid) is didecyldimethylammonium bromide, 1,2-dioleoyloxy-3-(trimethylammonium)propane, dioctyadecylamine, trimethyl[2,3-(dioleyloxy)propyl]ammonium, N-tert-butyl-N′-tetradecyl-3-(tetradecylamino)propanimidamide, cetrimonium, tridodecylamine, dimethyldioctadecylammonium, stearyltrimethylammonium, N,N-dimethyltetradecylamine, trioctylamine, cetrimide, dihexadecyl dimethyl ammonium, dimethyldipalmitylammonium, hexadecyldimethylamine, methyltrioctylammonium, dipalmitylamine, dimyristylamine, 1,2-di(oleolyoxy)-3-(dimethylamino)propane, or 2,5-bis(3-aminopropylamino)-N-[2-[di(heptadecyl)amino]-2-oxoethyl]pentanamide, ([(4-hydroxybutyl)azanediyl]di(hexane-6,1-diyl) bis(2-hexyldecanoate) (ALC-0315, 9-Heptadecanyl 8-{(2-hydroxyethyl)[6-oxo-6-(undecyloxy)hexyl]amino}octanoate (SM-102), N,N-dimethyl-2,3-dioleyloxy)propylamine (DODMA), N,N-dioleyl-N,N-dimethylammonium chloride (DODAC), N,N-distearyl-N,N-dimethylammonium bromide (DDAB), N-(1-(2,3-dioleoyloxy)propyl)-N,N,N-trimethylammonium chloride (DOTAP), N-(1-(2,3-dioleyloxy)propyl)-N,N,N-trimethylammonium chloride (DOTMA), 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-KC2-DMA), 2,2-dilinoleyl-4-dimethylaminomethyl-[1,3]-dioxolane (DLin-K-DMA), or a pharmaceutically acceptable salt thereof.

### Particle Compositions

The present disclosure features particles (e.g., lipid nanoparticles) and related compositions comprising a polysarcosine lipid conjugate (e.g., a compound of Formulas (I), (I-a), (I-b), (I-c), (I-d), (I-e), (I-f), or (I-g)) as described herein. Exemplary lipid nanoparticles include liposomes/niosomes, nanostructured lipid carriers, cationic lipid-nucleic acid complexes, and solid lipid nanoparticles. Details involving each of the foregoing particles are provided below in more detail.

### Lipid Nanoparticles

Described herein are lipid nanoparticle compositions comprising a polysarcosine lipid conjugate, e.g., a compound of Formula (I), (I-a), (I-b), (I-c), (I-d), (I-e), (I-f), or (I-g) as described herein. In some embodiments, the disclosure provides a particle comprising (i) a polysarcosine lipid conjugate (e.g., a polymer of Formulas (I), (I-a), (I-b), (I-c), (I-d), (I-e), (I-f), or (I-g)); (ii) a phospholipid (e.g., a compound of Formulas (II)-(II-e); (iii) a steroid (e.g., a compound of Formulas (III)-(III-b)); (iv) a payload (e.g., a nucleic acid or drug); and (v) an additional lipid component (e.g., a cationic lipid, e.g., ionizable lipid, e.g., a compound of Formulas (IV)-(IV-e)); wherein each of components (i)-(iv) are as described herein.

Exemplary lipid nanoparticle compositions are disclosed in International Patent Publication WO2010005721; U.S. Pat. No. 10,703,789; US20200254086, U.S. Pat. No. 11,406,706, and US20200345641, each of which is incorporated herein by reference in its entirety.

In some embodiments, the lipid nanoparticles comprise a polysarcosine lipid conjugate (e.g., a polymer of any of Formulas (I)-(I-g) as described herein), in an amount of 0.1 to 50 mol %, 0.1 to 45 mol %, 0.1 to 40 mol %, 0.1 to 35 mol %, 0.1 to 30 mol %, 0.1 to 20 mol %, 0.25 to 20 mol %, 0.5 to 20 mol %, 1 to 20 mol %, 1.5 to 20%, 0.1 to 15 mol %, 0.25 to 15 mol %, 0.5 to 15 mol %, 1 to 15 mol %, 1.5 to 15 mol %, 2 to 15 mol %, 2.5 to 15 mol %, 0.1 to 12.5 mol %, 0.25 to 12.5 mol %, 0.5 to 12.5 mol %, 1 to 12.5 mol %, 1.5 to 12.5 mol %, 2 to 12.5 mol %, 2.5 to 12.5 mol %, 0.1 to 10 mol %, 0.25 to 10 mol %, 0.5 to 10 mol %, 1 to 10 mol %, 1.5 to 10 mol %, 2 to 10 mol %, 2.5 to 10 mol %, 0.1 to 7.5 mol %, 0.25 to 7.5 mol %, 0.5 to 7.5 mol %, to 7.5 mol %, 1.5 to 7.5 mol %, 2 to 7.5 mol %, 2.5 to 7.5 mol %, 0.1 to 5 mol %, 0.25 to 5 mol %, 0.5 to 5 mol %, 1 to 5 mol %, 1.5 to 5 mol %, 2 to 5 mol %, 2.5 to 5 mol %, 0.1 to 3 mol %, 0.25 to 3 mol %, 0.5 to 3 mol %, 1 to 3 mol %, 1.5 to 3 mol %, 2 to 3 mol %, 2.5 to 3 mol %, 0.1 to 2.5 mol %, 0.25 to 2.5 mol %, 0.5 to 2.5 mol %, 1 to 2.5 mol %, 1.5 to 2.5 mol %, 2 to 2.5 mol %, 0.1 to 4.5 mol %, 0.25 to 4.5 mol %, 0.5 to 4.5 mol %, 1 to 4.5 mol %, 1.5 to 4.5 mol %, 2 to 4.5 mol %, 2.5 to 4.5 mol %, 0.1 to 4 mol %, 0.25 to 4 mol %, 0.5 to 4 mol %, 1 to 4 mol %, 1.5 to 4 mol %, 2 to 4 mol %, 2.5 to 4 mol %, 0.1 to 3.5 mol %, 0.25 to 3.5 mol %, 0.5 to 3.5 mol %, 1 to 3.5 mol %, 1.5 to 3.5 mol %, 2 to 3.5 mol %, 2.5 to 3.5 mol %, 0.1 to 2.0 mol %, 0.1 to 1.5 mol %, 0.1 to 1.0 mol %, 0.5 to 2.0 mol %, 0.5 to 1.5 mol %, 0.5 to 1.0 mol %, 1.0 to 2.0 mol %, 1.0 to 1.5 mol %, 1.5 to 2.0 mol %, 1.0 mol %, or 1.5 mol %.

In some embodiments, the lipid nanoparticles comprise a polysarcosine lipid conjugate (e.g., a polymer of any of Formulas (I)-(I-g) as described herein), in an amount greater than 70 mol %, greater than 65 mol %, greater than 60 mol %, greater than 55 mol %, greater than 50 mol %, greater than 45 mol %, greater than 40 mol %, greater than 35 mol %, greater than 30 mol %, greater than 25 mol %, greater than 20 mol percent, greater than 15 mol %, greater than 10 mol %, greater than 5 mol %, greater than 1 mol %, greater than 0.1 mol %. In some embodiments, the lipid nanoparticles comprise a polysarcosine lipid conjugate (e.g., a polymer of any of Formulas (I)-(I-g) as described herein), in an amount less than 70 mol %, less than 65 mol %, less than 60 mol %, less than 55 mol %, less than 50 mol %, less than 45 mol %, less than 40 mol %, less than 35 mol %, less than 30 mol %, less than 25 mol %, less than 20 mol percent, less than 15 mol %, less than 10 mol %, less than 5 mol %, less than 1 mol %, less than 0.1 mol %.

In some embodiments, the lipid nanoparticles comprise a phospholipid (e.g., a compound of Formulas (II)-(II-e) as described herein) in an amount of 1 to 75 mol %, 5 to 7 mol %, 10 to 75 mol %, 20 to 75 mol %, 35 to 75 mol %, 1 to 60 mol %, 5 to 60 mol %, 10 to 60 mol %, 20 to 60 mol %, 30 to 60 mol %, 1 to 50 mol %, 5 to 50 mol %, 5 to 40 mol %, 5 to 30 mol %, 5 to 25 mol %, 5 to 20 mol %, 5 to 15 mol %, 5 to 13 mol %, 5 to 10 mol %, 10 to 30 mol %, 10 to 25 mol %, 10 to 20 mol %, 10 to 15 mol %, 10 to 13 mol %, 15 to 30 mol %, 15 to 25 mol %, 15 to 20 mol %, 20 to 30 mol %, or 20 to 25 mol %.

In some embodiments, the lipid nanoparticles comprise a phospholipid (e.g., a compound of any of Formulas (II)-(II-e) as described herein), in an amount greater than 85 mol %, greater than 80 mol %, greater than 75 mol %, greater than 65 mol %, greater than 60 mol %, greater than 55 mol %, greater than 50 mol %, greater than 45 mol %, greater than 40 mol %, greater than 35 mol %, greater than 30 mol %, greater than 25 mol %, greater than 20 mol percent, greater than 15 mol %, greater than 10 mol %, greater than 5 mol %, greater than 1 mol %, greater than 0.1 mol %. In some embodiments, the lipid nanoparticles comprise a phospholipid (e.g., a compound of any of Formulas (II)-(II-e) as described herein), in an amount less than 80 mol %, less than 75 mol %, less than 70 mol %, less than 65 mol %, less than 60 mol %, less than 55 mol %, less than 50 mol %, less than 45 mol %, less than 40 mol %, less than 35 mol %, less than 30 mol %, less than 25 mol %, less than 20 mol percent, less than 15 mol %, less than 10 mol %, less than 5 mol %, less than 1 mol %, less than 0.1 mol %.

In some embodiments, the lipid nanoparticles comprise a steroid (e.g., cholesterol, e.g., a cholesterol derivative, e.g., a compound of Formulas (III)-(III-b) as described herein) in an amount of 10 to 60 mol %, 20 to 60 mol %, 30 to 60 mol %, 30 to 55 mol %, 30 to 52.5 mol %, 30 to 52 mol %, 30 to 51 mol %, 30 to 50 mol %, 30 to 47 mol %, 30 to 45 mol %, 30 to 44 mol %, 30 to 43 mol %, 30 to 43 mol %, 30 to 41 mol %, 30 to 40 mol %, 30 to 39 mol %, 35 to 60 mol %, 35 to 55 mol %, 35 to 52.5 mol %, 35 to 52 mol %, 35 to 51 mol %, 35 to 50 mol %, 35 to 47.5 mol %, 35 to 45 mol %, 35 to 44 mol %, 35 to 43.5 mol %, 35 to 43 mol %, 35 to 41.5 mol %, 35 to 40 mol %, 35 to 39 mol %, 37 to 60 mol %, 37 to 55 mol %, 37 to 52 mol %, 37 to 52 mol %, 37 to 51 mol %, 37 to 50 mol %, 37.5 to 47 mol %, 37 to 45 mol %, 37 to 44 mol %, 37 to 43 mol %, 37 to 43 mol %, 37.5 to 41 mol %, 37.5 to 40 mol %, 37.5 to 39.5 mol %, 39.5 to 60 mol %, 39.5 to 55 mol %, 39.5 to 52 mol %, 39 to 52 mol %, 39 to 51 mol %, 39 to 50 mol %, 39 to 47 mol %, 39 to 45 mol %, 39 to 44 mol %, 39 to 43 mol %, 39 to 43 mol %, 3 to 4 mol %, 39 to 40 mol %, 40 to 60 mol %, 40 to 55 mol %, 40 to 52 mol %, 40 to 52 mol %, 40 to 51 mol %, 40 to 50 mol %, 40 to 47 mol %, 40 to 45 mol %, 40 to 44 mol %, 40 to 43 mol %, 40 to 43 mol %, 40 to 41.5 mol %, 41 to 60 mol %, 41.5 to 55 mol %, 41.5 to 52 mol %, 41 to 52 mol %, 41 to 51 mol %, 41 to 50 mol %, 41 to 47 mol %, 41 to 45 mol %, 41 to 44 mol %, 41 to 43 mol %, 41 to 43 mol %, 43 to 60 mol %, 43 to 55 mol %, 43 to 52 mol %, 43 to 52 mol %, 43 to 51 mol %, 43 to 50 mol %, 43 to 47.5 mol %, 43 to 45 mol %, 43 to 44 mol %, 43 to 43 mol %, 43 to 60 mol %, 43 to 55 mol %, 43 to 52 mol %, 43 to 52 mol %, 43 to 51 mol %, 43 to 50 mol %, 43 to 47 mol %, 43 to 45 mol %, 43 to 44 mol %, 45 to 60 mol %, 45 to 55 mol %, 45 to 52 mol %, 45 to 52 mol %, 45 to 51 mol %, 45 to 50 mol %, 45 to 47 mol %, 47 to 60 mol %, 47 to 55 mol %, 47.5 to 52 mol %, 47 to 52 mol %, 47 to 51 mol %, 47 to 50 mol %, 50 to 60 mol %, 50 to 55 mol %, 50 to 52 mol %, 50 to 52 mol %, 50 to 52 mol % 50 to 51 mol %, 51 to 60 mol %, 51 to 55 mol %, 51 to 52.5 mol %, or 51 to 52 mol %, %, 51 to 60 mol %, 51 to 55 mol %, 51 to 52.5 mol %, or 51 to 52 mol %.

In some embodiments, the lipid nanoparticles comprise a steroid (e.g., cholesterol, e.g., a cholesterol derivative, e.g., a compound of Formulas (III)-(III-b) as described herein) in an amount greater than 70 mol %, greater than 65 mol %, greater than 60 mol %, greater than 55 mol %, greater than 50 mol %, greater than 45 mol %, greater than 40 mol %, greater than 35 mol %, greater than 30 mol %, greater than 25 mol %, greater than 20 mol percent, greater than 15 mol %, greater than 10 mol %, greater than 5 mol %, greater than 1 mol %, greater than 0.1 mol %. In some embodiments, the lipid nanoparticles comprise a steroid (e.g., cholesterol, e.g., a cholesterol derivative, e.g., a compound of Formulas (III)-(III-b) as described herein) in an amount less than 70 mol %, less than 65 mol %, less than 60 mol %, less than 55 mol %, less than 50 mol %, less than 45 mol %, less than 40 mol %, less than 35 mol %, less than 30 mol %, less than 25 mol %, less than 20 mol percent, less than 15 mol %, less than 10 mol %, less than 5 mol %, less than 1 mol %, less than 0.1 mol %.

In some embodiments, the lipid nanoparticles comprise an additional lipid component (e.g., cationic lipid, e.g., ionizable lipid, e.g., a compound of Formulas (IV)-(IV-c) as described herein) in an amount equal to (100−((mole percentage of polysarcosine lipid conjugate)+(mole percentage of steroid)+(mole percentage of phospholipid))) mole percentage. According to one example, the cationic or ionizable lipid may be comprised in the lipid nanoparticle in an amount of 10 to 70 mol %, 10 to 60 mol %, 10 to 55 mol %, 10 to 50 mol %, 10 to 45 mol %, 10 to 42.5 mol %, 10 to 40 mol %, 10 to 35 mol %, 10 to 30 mol %, 10 to 26.5 mol %, 10 to 25 mol %, 10 to 20 mol %, 15 to 60 mol %, 15 to 55 mol %, 15 to 50 mol %, 15 to 45 mol %, 15 to 42.5 mol %, 15 to 40 mol %, 15 to 35 mol %, 15 to 30 mol %, 15 to 26.5 mol %, 15 to 25 mol %, 15 to 20 mol %, 20 to 60 mol %, 20 to 55 mol %, 20 to 50 mol %, 20 to 45 mol %, 20 to 42.5 mol %, 20 to 40 mol %, 20 to 35 mol %, 20 to 30 mol %, 20 to 26.5 mol %, 20 to 25 mol %, 25 to 60 mol %, 25 to 55 mol %, 25 to 50 mol %, 25 to 45 mol %, 25 to 42.5 mol %, 25 to 40 mol %, 25 to 35 mol %, 25 to 30 mol %, 25 to 26.5 mol %, 26.5 to 60 mol %, 26.5 to 55 mol %, 26.5 to 50 mol %, 26.5 to 45 mol %, 26.5 to 42.5 mol %, 26.5 to 40 mol %, 26.5 to 35 mol %, 26.5 to 30 mol %, 30 to 60 mol %, 30 to 55 mol %, 30 to 50 mol %, 30 to 45 mol %, 30 to 42.5 mol %, 30 to 40 mol %, 30 to 35 mol %, 35 to 60 mol %, 35 to 55 mol %, 35 to 50 mol %, 35 to 45 mol %, 35 to 42.5 mol %, 35 to 40 mol %, 40 to 60 mol %, 40 to 55 mol %, 40 to 50 mol %, 40 to 45 mol %, 40 to 42.5 mol %, 42.5 to 60 mol %, 42.5 to 55 mol %, 42.5 to 50 mol %, or 42.5 to 45 mol %.

In some embodiments, the lipid nanoparticles comprise a additional lipid component (e.g., cationic lipid, e.g., ionizable lipid, e.g., a compound of Formulas (IV)-(IV-c) as described herein) in an amount greater than 70 mol %, greater than 65 mol %, greater than 60 mol %, greater than 55 mol %, greater than 50 mol %, greater than 45 mol %, greater than 40 mol %, greater than 35 mol %, greater than 30 mol %, greater than 25 mol %, greater than 20 mol percent, greater than 15 mol %, greater than 10 mol %, greater than 5 mol %, greater than 1 mol %, greater than 0.1 mol %. In some embodiments, the lipid nanoparticles comprise an additional lipid component (e.g., cationic lipid, e.g., ionizable lipid, e.g., a compound of Formulas (IV)-(IV-c) as described herein) in an amount less than 70 mol %, less than 65 mol %, less than 60 mol %, less than 55 mol %, less than 50 mol %, less than 45 mol %, less than 40 mol %, less than 35 mol %, less than 30 mol %, less than 25 mol %, less than 20 mol percent, less than 15 mol %, less than 10 mol %, less than 5 mol %, less than 1 mol %, less than 0.1 mol %.

In some embodiments, the lipid nanoparticles have a mean diameter of from about 20 nm to about 150 nm, from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, or larger, and are substantially non-toxic to a human subject.

In some embodiments, the mean diameter of the lipid nanoparticles is determined by dynamic light scattering. In some embodiments, the mean diameter of the lipid nanoparticles is determined by optical microscopy. In some embodiments, the mean diameter of the lipid nanoparticles is determined by wide-field or confocal microscopy. In some embodiments, the mean diameter is determined by size exclusion chromatography (SEC) or nuclear magnetic resonance (NMR) spectroscopy.

In some embodiments, the particles have a net charge or are neutral. In some embodiments, the particles have a net positive charge or net negative charge. In a preferred embodiment, the particles have a net positive charge. In some embodiments, the charge is determined by zeta potential measurements.

### Liposomes

Another exemplary particle composition comprising a polysarcosine lipid conjugate described herein is a liposome. Liposomes are lipid vesicles composed of a lipid bilayer (e.g., phospholipid bilayer), often with additional lipid components (e.g., cholesterol). Liposomes can be made from several different types of lipids, such as phospholipids, which are commonly found. Exemplary liposomal formulations are disclosed in US Patent No. 10, 722, 508; U.S. Pat. Nos. 10,220,095; and 11,071,713, each of which are incorporated herein by reference in their entirety.

Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

### Other Particles

Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

### Payloads

The present disclosure further provides particle compositions (e.g., lipid nanoparticles e.g., liposomes e.g., nanostructured lipid carriers) comprising a polysarcosine-lipid conjugate that further comprises a payload. Exemplary payloads include proteins (e.g., enzymes, antibodies, lipoproteins), natural or synthetic peptides, or peptides containing non-natural amino acids, nucleic acids (e.g., DNA or mRNA), or small molecule drugs. In an embodiment, the payload is a therapeutic agent, e.g., an agent useful for treatment of a disease, disorder, or condition.

In one embodiment, the payload is a protein. Exemplary proteins include hormones, enzymes, antibodies, cytokines, receptors, or variants and fragments thereof. For example, the payload may be tumor necrosis factor (TNF) alpha or beta; renin; colchicine; prolactin; corticotrophin; vasopressin; somatostatin; lypressin; pancreozymin; leuprolide; alpha-1-antitrypsin; clotting factors such as factor VIIIC, factor IX, tissue factor, and von Willebrands factor; anti-clotting factors such as Protein C; atrial natriuretic factor; lung surfactant; a plasminogen activator other than a tissue-type plasminogen activator (t-PA); bombesin; thrombin; hemopoietic growth factor; enkephalinase; RANTES (regulated on activation normally T-cell expressed and secreted); human macrophage inflammatory protein (MIP-1-alpha); a serum albumin such as human serum albumin; mullerian-inhibiting substance; relaxin A-chain; relaxin B-chain; prorelaxin; mouse gonadotropin-associated peptide; chorionic gonadotropin; a microbial protein, such as beta-lactamase; DNase; inhibin; activin; receptors for hormones or growth factors; integrin; protein A or D; rheumatoid factors; platelet-derived growth factor (PDGF); epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I), insulin-like growth factor binding proteins; CD proteins such as CD-3, CD-4, CD-8, and CD-19; erythropoietin; osteoinductive factors; immunotoxins; an interferon such as interferon-alpha (e.g., interferon.alpha.2A), -beta, -gamma, -lambda and consensus interferon; colony stimulating factors (CSFs), e.g., M-CSF, GM-CSF, and G-CSF; interleukins (TLs), e.g., IL-1 to IL-10; superoxide dismutase; T-cell receptors; surface membrane proteins; decay accelerating factor; transport proteins; homing receptors; addressins; fertility inhibitors such as the prostaglandins; fertility promoters; regulatory proteins; antibodies (including fragments thereof) and chimeric proteins, such as immunoadhesins; precursors, derivatives, prodrugs and analogues of these compounds, and pharmaceutically acceptable salts of these compounds, or their precursors, derivatives, prodrugs and analogues. Suitable proteins or peptides may be native or recombinant and include, e.g., fusion proteins.

Examples of protein payloads also include CCL1, CCL2 (MCP-1), CCL3 (MIP-1α), CCL4 (MIP-1β), CCL5 (RANTES), CCL6, CCL7, CCL8, CCL9 (CCL10), CCL11, CCL12, CCL13, CCL14, CCL15, CCL16, CCL17, CCL18, CCL19, CCL20, CCL21, CCL22, CCL23, CCL24, CCL25, CCL26, CCL27, CCL28, CXCL1 (KC), CXCL2 (SDF1a), CXCL3, CXCL4, CXCL5, CXCL6, CXCL7, CXCL8 (IL8), CXCL9, CXCL10, CXCL11, CXCL12, CXCL13, CXCL14, CXCL15, CXCL16, CXCL17, CX3CL1, XCL1, XCL2, TNFA, TNFB (LTA), TNFC (LTB), TNFSF4, TNFSF5 (CD40LG), TNFSF6, TNFSF7, TNFSF8, TNFSF9, TNFSF10, TNFSF11, TNFSF13B, EDA, IL2, IL15, IL4, IL13, IL7, IL9, IL21, IL3, IL5, TL6, IL11, IL27, IL30, IL31, OSM, LIF, CNTF, CTF1, IL12a, IL12b, IL23, IL27, IL35, IL14, IL16, IL32, IL34, IL10, IL22, IL19, IL20, IL24, IL26, IL29, IFNL1, IFNL2, IFNL3, IL28, IFNA1, IFNA2, IFNA4, IFNA5, IFNA6, IFNA7, IFNA8, IFNA10, IFNA13, IFNA14, IFNA16, IFNA17, IFNA21, IFNB1, IFNK, IFNW1, IFNG, IL1A (IL1F1), IL1B (IL1F2), IL1Ra (IL1F3), IL1F5 (IL36RN), IL1F6 (IL36A), IL1F7 (IL37), IL1F8 (IL36B), IL1F9 (IL36G), IL1F10 (IL38), IL33 (IL1F11), IL18 (IL1G), IL17, KITLG, IL25 (IL17E), CSF1 (M-CSF), CSF2 (GM-CSF), CSF3 (G-CSF), SPP1, TGFB1, TGFB2, TGFB3, CCL3L1, CCL3L2, CCL3L3, CCL4L1, CCL4L2, IL17B, IL17C, IL17D, IL17F, AIMP1 (SCYE1), MIF, Areg, BC096441, Bmp1, Bmp10, Bmp15, Bmp2, Bmp3, Bmp4, Bmp5, Bmp6, Bmp7, Bmp8a, Bmp8b, C1qtnf4, Ccl21a, Ccl27a, Cd70, Cer1, Cklf, Clcf1, Cmtm2a, Cmtm2b, Cmtm3, Cmtm4, Cmtm5, Cmtm6, Cmtm7, Cmtm8, Crlf1, Ctf2, Ebi3, Edn1, Fam3b, Fas1, Fgf2, Flt31, Gdf10, Gdf11, Gdf15, Gdf2, Gdf3, Gdf5, Gdf6, Gdf7, Gdf9, Gm12597, Gm13271, Gm13275, Gm13276, Gm13280, Gm13283, Gm2564, Gpi1, Grem1, Grem2, Grn, Hmgb1, Ifna11, Ifna12, Ifna9, Ifnab, Ifne, Il17a, Il23a, Il25, Il31, Iltifb, Inhba, Lefty1, Lefty2, Mstn, Nampt, Ndp, Nodal, Pf4, Pglyrp1, Pr17d1, Scg2, Scgb3al, Slurp1, Spp1, Thpo, Tnfsf10, Tnfsf11, Tnfsf12, Tnfsf13, Tnfsf13b, Tnfsf14, Tnfsf15, Tnfsf18, Tnfsf4, Tnfsf8, Tnfsf9, Tslp, Vegfa, Wnt1, Wnt2, Wnt5a, Wnt7a, Xcl1, Epinephrine, Melatonin, Triiodothyronine, Thyroxine, Prostaglandins, Leukotrienes, Prostacyclin, Thromboxane, Islet Amyloid Polypeptide, Müllerian inhibiting factor or hormone, Adiponectin, Corticotropin, Angiotensin, vasopressin, arginine vasopressin, atriopeptin, Brain natriuretic peptide, Calcitonin, Cholecystokinin, Cortistatin, Enkephalin, Endothelin, Erythropoietin, Follicle-stimulating hormone, Galanin, Gastric inhibitory polypeptide, Gastrin, Ghrelin, Glucagon, Glucagon-like peptide-1, Gonadotropin-releasing hormone, Growth hormone-releasing hormone, Hepcidin, Human chorionic gonadotropin, Human placental lactogen, Growth hormone, Inhibin, Insulin, Somatomedin, Leptin, Lipotropin, Luteinizing hormone, Melanocyte stimulating hormone, Motilin, Orexin, Oxytocin, Pancreatic polypeptide, Parathyroid hormone, Pituitary adenylate cyclase-activating peptide, Prolactin, Prolactin releasing hormone, Relaxin, Renin, Secretin, Somatostatin, Thrombopoietin, Thyrotropin, Thyrotropin-releasing hormone, Vasoactive intestinal peptide, Androgen, Androgen, acid maltase (alpha-glucosidase), glycogen phosphorylase, glycogen debrancher enzyme, Phosphofructokinase, Phosphoglycerate kinase, Phosphoglycerate mutase, Lactate dehydrogenase, Carnitine palymityl transferase, Carnitine, and Myoadenylate deaminase.

The protein payload may further be a hormone, for example, anti-diuretic hormone (ADH), which is produced by the posterior pituitary, targets the kidneys, and affects water balance and blood pressure; Oxytocin, which is produced by the posterior pituitary, targets the uterus, breasts, and stimulates uterine contractions and milk secretion; Growth Hormone (GH), which is produced by the anterior pituitary, targets the body cells, bones, muscles, and affects growth and development; Prolactin, which is produced by the anterior pituitary, targets the breasts, and maintains milk secretions; Growth Hormone-Releasing Hormone (GHRH), which is a releasing hormone of GH and is produced in the arcuate nucleas of the hypothalamus; Thyroid Stimulating Hormone (TSH), which is produced by the anterior pituitary, targets the thyroid, and regulates thyroid hormones; Thyrotropin-Release Hormone (TRH), which is produced by the hypothalamus and stimulates the release of TSH and prolactin from the anterior pituitary; Adrenocorticotropic Hormone (ACTH), which is produced by the anterior pituitary, targets the adrenal cortex, and regulates adrenal cortex hormones; Follicle-Stimulating Hormone (FSH), which is produced by the anterior pituitary, targets the ovaries/testes, and stimulates egg and sperm production; Lutenizing Hormone (LH), which is produced by the anterior pituitary, targets the ovaries/testes, and stimulates ovulation and sex hormone release; Luteinizing Hormone-Releasing Hormone (LHRH), also known as Gonadotropin-Releasing Hormone (GnRH), which is synthesized and released from GnRH neurons within the hypothalamus and is a trophic peptide hormone responsible for the release of FSH and LH; Thyroxine, which is produced by the thyroid, targets the body cells, and regulates metabolism; Calcitonin, which is produced by the thyroid, targets the adrenal cortex, and lowers blood calcium; Parathyroid Hormone, which is produced by the parathyroid, targets the bone matrix, and raises blood calcium; Aldosterone, which is produced by the adrenal cortex, targets the kidney, and regulates water balance; Cortisol, which is produced by the adrenal cortex, targets the body cells, and weakens immune system and stress responses; Epinephrine, which is produced by the adrenal medulla, targets the heart, lungs, liver, and body cells, and affects primary “fight or flight” responses; Glucagon, which is produced by the pancreas, targets the liver body, and raises blood glucose level; Insulin, which is produced by the pancreas, targets body cells, and lowers blood glucose level; Estrogen, which is produced by the ovaries, targets the reproductive system, and affects puberty, menstrual, and development of gonads; Progesterone, which is produced by the ovaries, targets the reproductive system, and affects puberty, menstrual cycle, and development of gonads; and Testosterone, which is produced by the adrenal gland, testes, targets the reproductive system, and affects puberty, development of gonads, and sperm.

In an embodiment, the protein payload is a growth hormone, such as human growth hormone (hGH), recombinant human growth hormone (rhGH), bovine growth hormone, methione-human growth hormone, des-phenylalanine human growth hormone, and porcine growth hormone; insulin, insulin A-chain, insulin B-chain, and proinsulin; or a growth factor, such as vascular endothelial growth factor (VEGF), nerve growth factor (NGF), platelet-derived growth factor (PDGF), fibroblast growth factor (FGF), epidermal growth factor (EGF), transforming growth factor (TGF), and insulin-like growth factor-I and -II (IGF-I and IGF-II).

In an embodiment, the protein payload is a vaccine component, such as an antigen. An antigen can include any protein or peptide that is foreign to the subject organism. Preferred antigens can be presented at the surface of antigen presenting cells (APC) of a subject for surveillance by immune effector cells, such as leucocytes expressing the CD4 receptor (CD4 T cells) and Natural Killer (NK) cells. Typically, the antigen is of viral, bacterial, protozoan, fungal, or animal origin. In some embodiments the antigen is a cancer antigen. Cancer antigens can be antigens expressed only on tumor cells and/or required for tumor cell survival.

Certain antigens are recognized by those skilled in the art as immuno-stimulatory (i.e., stimulate effective immune recognition) and provide effective immunity to the organism or molecule from which they derive. Antigens can be peptides, proteins, polysaccharides, saccharides, lipids, nucleic acids, or combinations thereof. The antigen can be derived from a virus, bacterium, parasite, plant, protozoan, fungus, tissue or transformed cell such as a cancer or leukemic cell and can be a whole cell or immunogenic component thereof, e.g., cell wall components or molecular components thereof. Suitable antigens are known in the art and are available from commercial government and scientific sources. The antigens may be purified or partially purified polypeptides derived from tumors or viral or bacterial sources. The antigens can be recombinant polypeptides produced by expressing DNA encoding the polypeptide antigen in a heterologous expression system. The antigens can be DNA encoding all or part of an antigenic protein. Antigens may be provided as single antigens or may be provided in combination. Antigens may also be provided as complex mixtures of polypeptides or nucleic acids.

Exemplary antigens may be isolated from any virus including, but not limited to, a virus from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxviridae (e.g., vaccinia and smallpox virus), Reoviridae (e.g., rotavirus), Retroviridae (e.g., lentivirus, such as human immunodeficiency virus (HIV) 1 and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3. Viral antigens may be derived from a particular strain such as a papilloma virus, a herpes virus, i.e. herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

Additional exemplary antigens can originate from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia. Additional exemplary antigens may include antigens can be obtained from parasites such as, but not limited to, an antigen derived from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Circumsporozoite protein, a Sporozoite surface protein, a liver stage antigen, an apical membrane associated protein, or a Merozoite surface protein.

Still further exemplary antigens can be an allergen or environmental antigen, such as, but not limited to, an antigen derived from naturally occurring allergens such as pollen allergens (tree-, herb, weed-, and grass pollen allergens), insect allergens (inhalant, saliva and venom allergens), animal hair and dandruff allergens, and food allergens. Important pollen allergens from trees, grasses and herbs originate from the taxonomic orders of Fagales, Oleales, Pinales and platanaceae including i.a. birch (Betula), alder (Alnus), hazel (Corylus), hornbeam (Carpinus) and olive (Olea), cedar (Cryptomeria and Juniperus), Plane tree (Platanus), the order of Poales including i.e. grasses of the genera Lolium, Phleum, Poa, Cynodon, Dactylis, Holcus, Phalaris, Secale, and Sorghum, the orders of Asterales and Urticales including herbs of the genera Ambrosia, Artemisia, and Parietaria. Other allergen antigens that may be used include allergens from house dust mites of the genus Dermatophagoides and Euroglyphus, storage mite e.g. Lepidoglyphys, Glycyphagus and Tyrophagus, those from cockroaches, midges and fleas e.g. Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

Additional exemplary antigens may include a tumor antigen, including a tumor-associated or tumor-specific antigen, such as, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARa fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, pi85erbB2, pi80erbB-3, c-met, nm-23H1, PSA, TAG-72-4, CA 19-9, CA 72-4, CAM 17.1, NuMa, K-ras, b-Catenin, CDK4, Mum-1, p16, TAGE, PSMA, PSCA, CT7, telomerase, 43-9F, 5T4, 791Tgp72, a-fetoprotein, 13HCG, BCA225, BTAA, CA 125, CA 15-3 (CA 27.29BCAA), CA 195, CA 242, CA-50, CAM43, CD68KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS.

In another embodiment, the payload is an antibody. Antibodies that function by binding directly to one or more epitopes, other ligands or accessory molecules at the surface of eukaryote cells, are described. Typically, the antibody or antigen binding fragment thereof has affinity for a receptor at the surface of a specific cell type, such as a receptor expressed at the surface of macrophage cells. Various types of antibodies and antibody fragments can be used in the described compositions and methods, including whole immunoglobulin of any class, fragments thereof, and synthetic proteins containing at least the antigen binding variable domain of an antibody. The antibody can be an IgG antibody, such as IgG₁, IgG₂, IgG₃, or IgG₄. An antibody can be in the form of an antigen binding fragment including a Fab fragment, F(ab′)2 fragment, a single chain variable region, and the like. Antibodies can be polyclonal or monoclonal (mAb). Monoclonal antibodies include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity (U.S. Pat. No. 4,816,567; and Morrison, et al., Proc. Natl. Acad. Sci. USA, 81: 6851-6855 (1984)). The described antibodies can also be modified by recombinant means, for example by deletions, additions or substitutions of amino acids, to increase efficacy of the antibody in mediating the desired function. Substitutions can be conservative substitutions. For example, at least one amino acid in the constant region of the antibody can be replaced with a different residue (see, e.g., U.S. Pat. Nos. 5,624,821; 6,194,551; WO 9958572; and Angal, et al., Mol. Immunol. 30:105-08 (1993)). In some cases changes are made to reduce undesired activities, e.g., complement-dependent cytotoxicity. The antibody can be a bi-specific antibody having binding specificities for at least two different antigenic epitopes. In one embodiment, the epitopes are from the same antigen. In another embodiment, the epitopes are from two different antigens. Bi-specific antibodies can include bi-specific antibody fragments (see, e.g., Hollinger, et al., Proc. Natl. Acad. Sci. U.S.A., 90:6444-48 (1993); Gruber, et al., J. Immunol., 152:5368 (1994)).

Antibodies can be generated by any means known in the art. Exemplary descriptions means for antibody generation and production include Delves, Antibody Production: Essential Techniques (Wiley, 1997); Shephard, et al., Monoclonal Antibodies (Oxford University Press, 2000); Goding, Monoclonal Antibodies: Principles And Practice (Academic Press, 1993); and Current Protocols In Immunology (John Wiley & Sons, most recent edition). Fragments of intact Ig molecules can be generated using methods well known in the art, including enzymatic digestion and recombinant means.

In some embodiments, the payload is a small molecule drug. In some embodiments, the small molecule drug is cytotoxic agent, chemotherapeutic agent, natural product, anti-viral, antibiotic or other therapeutic agents. Cytotoxic agents may include, for example, SN-38, paclitaxel, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, rachelmycin, amiodarone, isavuconazonium, delafloxacin, remdesivir, carfilzomib, posaconazole, allopregnanolone, dalbavancin, plerixafor, netupitant, erbulin, letermovir, palonosetron, copanlisib, lurbinectedin, and analogs thereof. Other therapeutic agents may include, for example, antimetabolites (e.g., methotrexate, 6-mercaptopurine, 6-thioguanine, cytarabine, and 5-fluorouracil, and decarbazine), alkylating agents (e.g., mechlorethamine, thiotepa, chlorambucil, rachelmycin, melphalan, carmustine, lomustine, cyclophosphamide, busulfan, dibromomannitol, streptozotocin, mitomycin C, and cis-dichlorodiamine platinum (II) (DDP), and cisplatin), anthracyclines (e.g., daunorubicin and doxorubicin), antibiotics (e.g., dactinomycin, bleomycin, mithramycin, and anthramycin), and anti-mitotic agents (e.g., vincristine, vinblastine, and maytansinoids).

In some embodiments, the particle compositions (e.g., lipid nanoparticles) comprise a payload selected from a therapeutic agent described in U.S. Pat. No. 8,734,846; or US Patent Publication US20100087337.

### EXEMPLARY ENUMERATED EMBODIMENTS

1. A particle comprising:
(i) a polymer of Formula (I-d):

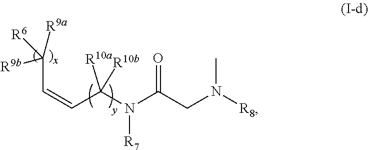

or a pharmaceutically acceptable salt thereof, wherein each of R⁶, R⁷, and R⁸ is independently hydrogen, heteroalkyl, alkyl, alkenyl, or alkynyl; each of R^{9a}, R^{9b}, R^{10a}, and R^{10b} is independently hydrogen, alkyl, or halo; each of x and y is independently an integer between 1 and 20; and z is an integer between 5 and 90; and one or more of:
(ii) a phospholipid;
(iii) a steroid;
(iv) a payload; and
(v) an additional lipid component.

2. The particle of embodiment 1, comprising (ii).

3. The particle of any one of the preceding embodiments, comprising (iii).

4. The particle of any one of the preceding embodiments, comprising (iv).

5. The particle of any one of the preceding embodiments, comprising (v).

6. The particle of any one of the preceding embodiments, comprising (ii), and (iii).

7. The particle of any one of the preceding embodiments, comprising (ii), (iii), and (iv).

8. The particle of any one of the preceding embodiments, comprising (ii), (iii), (iv), and (v).

9. The particle of any one of the preceding embodiments, comprising (ii), (iii), and (v).

10. The particle of any one of the preceding embodiments, comprising (ii), (iv), and (v).

11. The particle of any one of the preceding embodiments, comprising (ii) and (iii).

12. The particle of any one of the preceding embodiments, which does not comprise (iii).

13. The particle of any one of the preceding embodiments, which does not comprise (iv).

14. The particle of any one of the preceding embodiments, which does not comprise (v).

15. The particle of any one of the preceding embodiments, wherein each of R⁶, R⁷, and R⁸ is independently hydrogen.

16. The particle of any one of the preceding embodiments, wherein R⁶ is hydrogen.

17. The particle of any one of the preceding embodiments, wherein R⁷ is hydrogen.

18. The particle of any one of the preceding embodiments, wherein R⁸ is hydrogen.

19. The particle of any one of the preceding embodiments, wherein each of R^{9a}, R^{9b}, R^{10a}, and R^{10b} is independently hydrogen.

20. The particle of any one of the preceding embodiments, wherein R^{9a} is hydrogen.

21. The particle of any one of the preceding embodiments, wherein R^{9b} is hydrogen.

22. The particle of any one of the preceding embodiments, wherein R^{10a} is hydrogen.

23. The particle of any one of the preceding embodiments, wherein R^{10b} is hydrogen.

24. The particle of any one of the preceding embodiments, wherein z is 5-50.

25. The particle of any one of the preceding embodiments, wherein z is 5-48, 5-45, 5-40, 5-35, 5-30, 5-25, 5-20, 5-18, or 5-15.

26. The particle of any one of the preceding embodiments, wherein z is 15.

27. The particle of any one of the preceding embodiments, wherein z is 25.

28. The particle of any one of the preceding embodiments, wherein z is 30.

29. The particle of any one of the preceding embodiments, wherein z is 35.

30. The particle of any one of the preceding embodiments, wherein z is 40.

31. The particle of any one of the preceding embodiments, wherein z is 45.

32. The particle of any one of the preceding embodiments, wherein x is between 5-10.

33. The particle of any one of the preceding embodiments, wherein x is between 6-8.

34. The particle of any one of the preceding embodiments, wherein x is 5.

35. The particle of any one of the preceding embodiments, wherein x is 6.

36. The particle of any one of the preceding embodiments, wherein x is 7.

37. The particle of any one of the preceding embodiments, wherein x is 8.

38. The particle of any one of the preceding embodiments, wherein x is 9.

39. The particle of any one of the preceding embodiments, wherein x is 10.

40. The particle of any one of the preceding embodiments, wherein y is between 5-10.

41. The particle of any one of the preceding embodiments, wherein y is between 6-8.

42. The particle of any one of the preceding embodiments, wherein y is 5.

43. The particle of any one of the preceding embodiments, wherein y is 6.

44. The particle of any one of the preceding embodiments, wherein y is 7.

45. The particle of any one of the preceding embodiments, wherein y is 8.

46. The particle of any one of the preceding embodiments, wherein y is 9.

47. The particle of any one of the preceding embodiments, wherein y is 10.

48. The particle of any one of the preceding embodiments, wherein the polymer of Formula (I-d) is present at a mole percent of about 1% to 60%, 1% to 55%, 1% to 50%, 1% to 45%, 1% to 40%, 1% to 35%, 1% to 30%, 1% to 25%, 1% to 20%, 1% to 15%, 1% to 10%, 1% to 5%, 1% to 4%, 1% to 3%, 1% to 2%, 0.5%-2%, or 0.5% to 1%.

49. The particle of any one of the preceding embodiments, wherein the polymer of Formula (I-d) is present at a mole percent of about 1% to 60%.

50. The particle of any one of the preceding embodiments, wherein the polymer of Formula (I-d) is present at a mole percent of about 1% to 20%.

51. The particle of any one of the preceding embodiments, wherein the polymer of Formula (I-d) is present at a mole percent of about 1% to 10%.

52. The particle of any one of the preceding embodiments, wherein the polymer of Formula (I-d) is present at a mole percent of about 1% to 5%.

53. The particle of any one of the preceding embodiments, wherein the polymer of Formula (I-d) is present at a mole percent of about 1% to 4%.

54. The particle of any one of the preceding embodiments, wherein the polymer of Formula (I-d) is present at a mole percent of about 1% to 3%.

55. The particle of any one of the preceding embodiments, wherein the polymer of Formula (I-d) is present at a mole percent of about 1% to 2%.

56. The particle of any one of the preceding embodiments, wherein the polymer of Formula (I-d) is present at a mole percent of about 0.5% to 2%.

57. The particle of any one of the preceding embodiments, wherein the polymer of Formula (I-d) is present at a mole percent of about 1% to 15%.

58. The particle of any one of the preceding embodiments, wherein the polymer of Formula (I-d) is present at a mole percent of about 1.5%.

59. The particle of any one of the preceding embodiments, wherein the polymer of Formula (I-d) is present at a mole percent of about 2.5%.

60. The particle of any one of the preceding embodiments, wherein the particle is a liposome, noisome, or lipid nanoparticle.

61. The particle of any one of the preceding embodiments, wherein the particle is a liposome.

62. The particle of any one of the preceding embodiments, wherein the particle is a noisome.

63. The particle of any one of the preceding embodiments, wherein the particle is a lipid nanoparticle.

64. The particle of any one of the preceding embodiments, wherein the phospholipid is selected from a phosphatidylcholine, phosphatidylethanolamine, phosphatidylserine, and phosphatidylglycerol.

65. The particle of any one of the preceding embodiments, wherein the phospholipid is a phosphatidylcholine.

66. The particle of any one of the preceding embodiments, wherein the phospholipid is a phosphatidylethanolamine.

67. The particle of any one of the preceding embodiments, wherein the phospholipid is a phosphatidylserine.

68. The particle of any one of the preceding embodiments, wherein the phospholipid is a phosphatidylglycerol.

69. The particle of any one of the preceding embodiments, wherein the phospholipid is a compound of any one of Formulas (II)-(II-d), e.g., as described herein.

70. The particle of any one of the preceding embodiments, wherein the phospholipid is a compound of Formula (II):

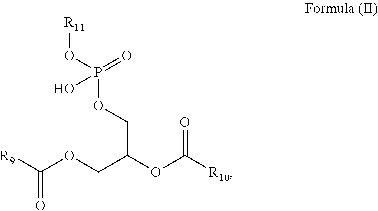

or a pharmaceutically acceptable salt thereof, wherein each R⁹ and R¹⁰ is independently alkyl, alkenyl, or alkynyl, optionally substituted with one or more R³; R¹¹ is alkyl, alkenyl, alkynyl, or heteroalkyl, optionally substituted with one or more R¹²; R³ is hydrogen, halo, oxo, cyano, nitro, —OR^{C}, alkyl, alkenyl, alkynyl, heteroalkyl, cycloalkyl, heterocyclyl, aryl, or heteroaryl, optionally substituted with one or more R⁴; R^{C} is hydrogen, alkyl, alkenyl, alkynyl, or heteroalkyl; R⁴ is halo, alkyl, heteroalkyl, or —OR^{C}; R¹² is alkyl, alkenyl, alkynyl, —NR^{D}R^{E}, oxo, or hydroxy; and each of R^{D} and R^{E} is independently hydrogen or alkyl.

71. The particle of embodiment 70, wherein for Formula (II), R⁹ is alkyl; R¹⁰ is alkyl; and R¹¹ is heteroalkyl.

72. The particle of embodiment 70 or 71, wherein for Formula (II), R⁹ is C17 alkyl; R¹⁰ is C17 alkyl; and R¹¹ is heteroalkyl (e.g., —CH₂CH₃N(CH₃)₃).

73. The particle of any one of embodiments 70-72, wherein the phospholipid of Formula (II) is

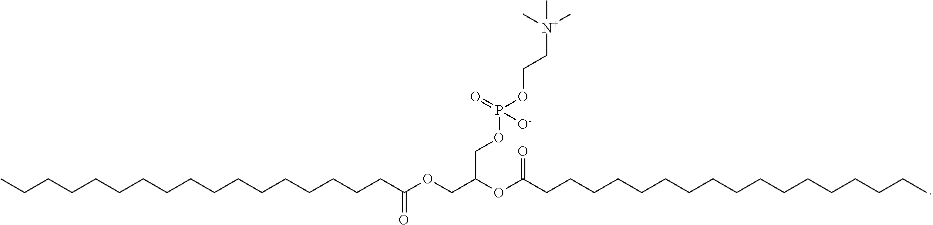 [SMILES: CCCCCCCCCCCCCCCCCC(=O)OCC(COP(=O)([O-])OCC[N+](C)(C)C)OC(=O)CCCCCCCCCCCCCCCCC]

74. The particle of any one of the preceding embodiments, wherein the phospholipid is a compound of Formulas (II-a) as described herein.

75. The particle of embodiment 74, wherein for Formula (II-a), R⁹ is alkyl; R¹⁰ is alkyl; R¹³ is alkyl; R¹⁴ is alkyl; and R¹⁵ is alkyl.

76. The particle of embodiment 74 or 75, wherein for Formula (II-a), R⁹ is C17 alkyl; R¹⁰ is C17 alkyl; R¹³ is C1 alkyl; R¹⁴ is C1 alkyl; and R¹⁵ is C1 alkyl.

77. The particle of any one of the preceding embodiments, wherein the phospholipid is a compound of Formula (II-b) as described herein.

78. The particle of any one of the preceding embodiments, wherein the phospholipid is a compound of Formula (II-c) as described herein.

79. The particle of any one of the preceding embodiments, wherein the phospholipid is a compound of Formula (II-d) as described herein.

80. The particle of any one of the preceding embodiments, wherein the phospholipid is a 1,2-dilinoleoyl-sn-glycero-3-phosphocholine (DLPC).

81. The particle of any one of the preceding embodiments, wherein the phospholipid is 1,2-dimyristoyl-sn-glycero-phosphocholine (DMPC).

82. The particle of any one of the preceding embodiments, wherein the phospholipid is 1,2-dioleoyl-sn-glycero-3-phosphocholine (DOPC).

83. The particle of any one of the preceding embodiments, wherein the phospholipid is 1,2-dipalmitoyl-sn-glycero-3-phosphocholine (DPPC).

84. The particle of any one of the preceding embodiments, wherein the phospholipid is 1,2-distearoyl-sn-glycero-3-phosphocholine (DSPC).

85. The particle of any one of the preceding embodiments, wherein the phospholipid is 1,2-diundecanoyl-sn-glycero-phosphocholine (DUPC).

86. The particle of any one of the preceding embodiments, wherein the phospholipid is 1-palmitoyl-2-oleoyl-sn-glycero-3-phosphocholine (POPC).

87. The particle of any one of the preceding embodiments, wherein the phospholipid further comprises a polyethylene glycol modification.

88. The particle of embodiment 87, wherein the phospholipid comprising a polyethylene glycol modification is selected from: methoxypolyethylene-glycoloxy(2000)-N,N-ditetradecylacetamide (ALC-0159), 1,2-dimyristoyl-rac-glycero-3-methoxypolyethylene glycol-2000 (DMG-PEG 2000), 1,2-dimyristoyl-rac-glycero-3-methoxypolyethylene glycol-5000 (DMG-PEG 5000), 1,2-dioleoyl-sn-glycero-3-phosphoethanolamine-N-[amino(polyethylene glycol)-2000] (DOPE-PEG(2000), 1,2-dioleoyl-sn-glycero-3-phosphoethanolamine-N-[amino(polyethylene glycol)-5000] (DOPE-PEG(5000), 1,2-distearoyl-sn-glycero-3-phosphoethanolamine-N-[amino(polyethylene glycol)-2000] (DSPE-PEG(2000), 1,2-distearoyl-sn-glycero-3-phosphoethanolamine-N-[amino(polyethylene glycol)-5000] (DSPE-PEG(5000), distearoyl-rac-glycerol-PEG2K (DSG-PEG 2000), and distearoyl-rac-glycerol-PEG5K (DSG-PEG 5000).

89. The particle of embodiment 87 or 88, wherein the phospholipid comprising a polyethylene glycol modification is selected from: 1,2-dimyristoyl-rac-glycero-3-methoxypolyethylene glycol-2000 (DMG-PEG 2000), 1,2-dioleoyl-sn-glycero-3-phosphoethanolamine-N-[amino(polyethylene glycol)-2000] (DOPE-PEG(2000), 1,2-distearoyl-sn-glycero-3-phosphoethanolamine-N-[amino(polyethylene glycol)-2000] (DSPE-PEG(2000), and distearoyl-rac-glycerol-PEG2K (DSG-PEG 2000).

90. The particle of embodiment 97 or 88, wherein the phospholipid comprising a polyethylene glycol modification is 1,2-dimyristoyl-rac-glycero-3-methoxypolyethylene glycol-2000 (DMG-PEG 2000).

91. The particle of any one of the preceding embodiments, wherein phospholipid is a combination of one or more phospholipids described herein.

92. The particle of any one of the preceding embodiments, wherein the steroid is a compound of any one of Formulas (III)-(III-b), e.g., as described herein.

93. The particle of any one of the preceding embodiments, wherein the steroid is a compound of Formula (III as described herein.

94. The particle of embodiment 90, wherein for Formula (III): ring positions 3, 10, 13, and 17 are each substituted with 1 R³; and the bond between ring positions 5 and 6 is a double bond.

95. The particle of any one of the preceding embodiments, wherein the steroid is a compound of Formula (III-a) as described herein.

96. The particle of embodiment 95, wherein for Formula (III-a): R²¹ is —OR^{C}; R²⁴ is alkyl; R²⁵ is alkyl; R²⁶ is alkyl substituted with two R³; and R²², R²³, and R²⁷ are hydrogen.

97. The particle of embodiment 95 or 96, wherein for Formula (III-a): R²¹ is —OH; R²⁴ is methyl; R²⁵ is methyl; R²⁶ is heptyl substituted with two R³; and R²², R²³, and R²⁷ are hydrogen.

98. The particle of any one of the preceding embodiments, wherein the steroid is a compound of Formula (III-b) as described herein.

99. The particle of embodiment 98, wherein for Formula (III-b): R³¹ is —OR^{A}; R³² is alkyl; R³³ is alkyl; R³⁴ is alkyl; R³⁵ is alkyl; and R³⁶ is alkyl.

100. The particle of any one of the preceding embodiments, wherein the steroid is cholesterol or a cholesterol derivative.

101. The particle of any one of the preceding embodiments, wherein the steroid is cholesterol.

102. The particle of any one of the preceding embodiments, wherein the payload is selected from a protein, enzyme, peptide, nucleic acid, small molecule, or lipid.

103. The particle of any one of the preceding embodiments, wherein the payload is a protein.

104. The particle of any one of the preceding embodiments, wherein the payload is an enzyme, peptide.

105. The particle of any one of the preceding embodiments, wherein the payload is peptide.

106. The particle of any one of the preceding embodiments, wherein the payload is nucleic acid.

107. The particle of any one of the preceding embodiments, wherein the payload is small molecule.

108. The particle of any one of the preceding embodiments, wherein the payload is lipid.

109. The particle of embodiment 102, wherein the nucleic acid is an RNA molecule.

110. The particle of embodiment 105, wherein the RNA molecule is selected from: mRNA, tRNA, rRNA, and snRNA.

111. The particle of embodiment 105 or 106, wherein the RNA molecule is an mRNA molecule.

112. The particle of embodiment 102, wherein the payload is a small molecule selected from a cytotoxic agent, chemotherapeutic agent, natural product, antiviral, and an antibiotic.

113. The particle of embodiment 112, wherein the small molecule is a cytotoxic agent.

114. The particle of embodiment 112, wherein the small molecule is a chemotherapeutic agent.

115. The particle of embodiment 112, wherein the small molecule is a natural product.

116. The particle of embodiment 112, wherein the small molecule is an antiviral.

117. The particle of embodiment 112, wherein the small molecule is an antibiotic.

118. The particle of any one of the preceding embodiments, wherein the payload is selected from SN-38, paclitaxel, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, a glucocorticoid compound, procaine, tetracaine, lidocaine, propranolol, puromycin, rachelmycin, amiodarone, isavuconazonium, delafloxacin, remdesivir, carfilzomib, posaconazole, allopregnanolone, dalbavancin, plerixafor, netupitant, erbulin, letermovir, palonosetron, copanlisib, lurbinectedin, methotrexate, 6-mercaptopurine, 6-thioguanine, cytarabine, 5-fluorouracil, decarbazine, mechlorethamine, thiotepa, chlorambucil, rachelmycin, melphalan, carmustine, lomustine, cyclophosphamide, busulfan, dibromomannitol, streptozotocin, mitomycin C, cis-dichlorodiamine platinum (II), cisplatin, daunorubicin, doxorubicin, dactinomycin, bleomycin, mithramycin, anthramycin, vincristine, vinblastine, and maytansinoid.

119. The particle of any one of the preceding embodiments, wherein the additional lipid component is a cationic lipid.

120. The particle of any one of the preceding embodiments, wherein the additional lipid component is a compound of Formulas (IV)-(IV-b), e.g., as described herein.

121. The particle of any one of the preceding embodiments, wherein the additional lipid component is a compound of Formula (IV).

122. The particle of any one of the preceding embodiments, wherein the additional lipid component is SM-102.

123. The particle of any one of the preceding embodiments, wherein the additional lipid component is ALC-0315.

124. A lipid nanoparticle comprising:
(i) a polymer of Formula (I-d):

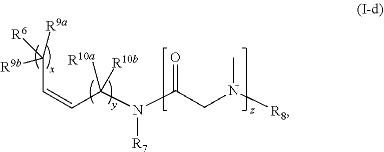

or a pharmaceutically acceptable salt thereof, wherein each of R⁶, R⁷, and R⁸ is independently hydrogen, heteroalkyl, alkyl, alkenyl, or alkynyl; each of R^{9a}, R^{9b}, R^{10a}, and R^{10b} is independently hydrogen, alkyl, or halo; each of x and y is independently an integer between 1 and 20; and z is an integer between 5 and 90; and one or more of:
(ii) a phospholipid;
(iii) a steroid;
(iv) a payload; and
(v) an additional lipid component.

125. A lipid nanoparticle comprising:
(i) a polymer of Formula (I-d):

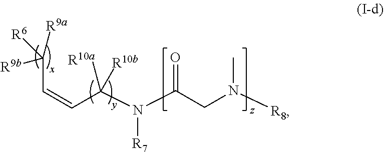

or a pharmaceutically acceptable salt thereof, wherein each of R⁶, R⁷, and R⁸ is independently hydrogen, heteroalkyl, alkyl, alkenyl, or alkynyl; each of R^{9a}, R^{9b}, R^{10a}, and R^{10b} is independently hydrogen, alkyl, or halo; each of x and y is independently an integer between 1 and 20; and z is an integer between 5 and 90:
(ii) a phospholipid;
(iii) a steroid;
(iv) a payload; and
(v) an additional lipid component.

126. A lipid nanoparticle comprising:
(i) a polymer having the structure:

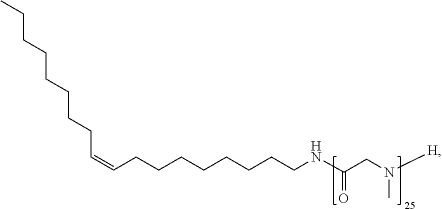

or a pharmaceutically acceptable salt thereof,
and one or more of:
(ii) a phospholipid;
(iii) a steroid;
(iv) a payload; and
(v) an additional lipid component.

127. A lipid nanoparticle comprising:
(i) a polymer having the structure:

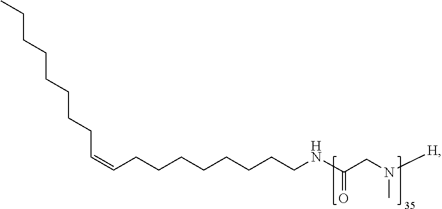

or a pharmaceutically acceptable salt thereof,
(ii) a phospholipid;
(iii) a steroid;
(iv) a payload; and
(v) an additional lipid component.

128. A lipid nanoparticle comprising:
(i) a polymer having the structure:

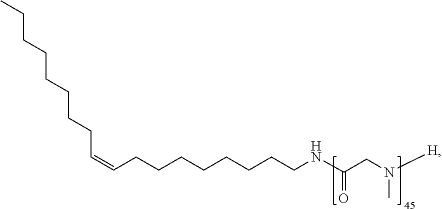

or a pharmaceutically acceptable salt thereof,
(ii) a phospholipid;
(iii) a steroid;
(iv) a payload; and
(v) an additional lipid component.

129. The particle or lipid nanoparticle of any one of the preceding embodiments, wherein the phospholipid comprises diastearoylphosphatidylcholine (DSPC) or 1,2-dimyristoyl-rac-glycero-3-methoxypolyethylene glycol (DMG-PEG).

130. The particle or lipid nanoparticle of embodiment 129, wherein the DMG-PEG comprises DMG-PEG-2000, DMG-PEG-3000, DMG-PEG-3350, DMG-PEG-4000, DMG-PEG-5000, DMG-PEG-10,000, or DMG-PEG-20,000.

131. The particle or lipid nanoparticle of embodiment 129 or 130, wherein the DMG-PEG comprises DMG-PEG-2000.

132. The particle or lipid nanoparticle of embodiment 129 or 130, wherein the DMG-PEG comprises DMG-PEG-3000.

133. The particle or lipid nanoparticle of embodiment 129 or 130, wherein the DMG-PEG comprises DMG-PEG-3350.

134 The particle or lipid nanoparticle of embodiment 129 or 130, wherein the DMG-PEG comprises DMG-PEG-4000.

135. The particle or lipid nanoparticle of embodiment 129 or 130, wherein the DMG-PEG comprises DMG-PEG-5000.

136. The particle or lipid nanoparticle of embodiment 129 or 130, wherein the DMG-PEG comprises DMG-PEG-10,000.

137. The particle or lipid nanoparticle of embodiment 129 or 130, wherein the DMG-PEG comprises DMG-PEG-20,000.

138. The particle or lipid nanoparticle of any one of the preceding embodiments, wherein the particle or lipid nanoparticle comprises a plurality of phospholipids.

139. The particle or lipid nanoparticle of any one of the preceding embodiments, wherein the particle comprises DSPC and a DMG-PEG.

140. The particle or lipid nanoparticle of embodiment 139, wherein the concentration of DSPC provided in the particle or lipid nanoparticle formulation is between 5-15%.

141. The particle or lipid nanoparticle of any one of embodiments 139-140, wherein the concentration of DMG-PEG provided in the particle or lipid nanoparticle formulation is between 0.5-5%.

142. The particle or lipid nanoparticle of any one of embodiments 129-141, wherein the concentration of DMG-PEG-2,000 provided in the particle or lipid nanoparticle formulation is between 0.5-5%.

143. The particle of any one of the preceding embodiments, wherein the polymer of Formula (I-d) is selected from oleyl-pSar10, oleyl-pSar15, and oleyl-pSar30.

144. The particle of any one of the preceding embodiments, wherein the polymer of Formula (I-d) is oleyl-pSar10.

145. The particle of any one of the preceding embodiments, wherein the polymer of Formula (I-d) is oleyl-pSar15.

146. The particle of any one of the preceding embodiments, wherein the polymer of Formula (I-d) is oleyl-pSar30.

147. The particle or lipid nanoparticle of any one of the preceding embodiments, wherein the concentration of polymer of Formula (I-d) is between 0.5-5%.

148. The particle or lipid nanoparticle of any one of the preceding embodiments, wherein the diameter of the particle or lipid nanoparticle is between 50 nm-1 mm.

149. The particle or lipid nanoparticle of any one of the preceding embodiments, wherein the diameter of the particle or lipid nanoparticle is between 50 nm-750 nm.

150. The particle or lipid nanoparticle of any one of the preceding embodiments, wherein the diameter of the particle or lipid nanoparticle is between 50 nm-500 nm.

151. The particle or lipid nanoparticle of any one of the preceding embodiments, wherein the diameter of the particle or lipid nanoparticle is between 50-250 nm.

152. The particle or lipid nanoparticle of any one of the preceding embodiments, wherein the diameter of the particle or lipid nanoparticle is between 50 nm-200 nm.

153. The particle or lipid nanoparticle of any one of the preceding embodiments, wherein the diameter of the particle or lipid nanoparticle is between 50 nm-150 nm.

154. The particle or lipid nanoparticle of any one of the preceding embodiments, wherein the polydispersity of the particle or lipid nanoparticle is between 0.05-0.5.

155. The particle or lipid nanoparticle of any one of the preceding embodiments, wherein the polydispersity of the particle or lipid nanoparticle is between 0.1-0.4.

156. The particle or lipid nanoparticle of any one of the preceding embodiments, wherein the polydispersity of the particle or lipid nanoparticle is between 0.1-0.3.

157. The particle or lipid nanoparticle of any one of the preceding embodiments, wherein the polydispersity of the particle or lipid nanoparticle is between 0.1-0.2.

158. A method of delivering a payload to a subject or cell, the method comprising administering to the subject or cell a particle comprising:
(i) a polymer of Formula (I-d):

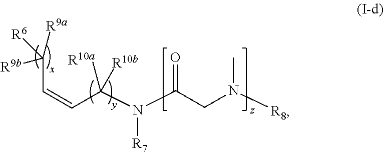

or a pharmaceutically acceptable salt thereof, wherein each of R⁶, R⁷, and R⁸ is independently hydrogen, heteroalkyl, alkyl, alkenyl, or alkynyl; each of R^{9a}, R^{9b}, R^{10a}, and R^{10b} is independently hydrogen, alkyl, or halo; each of x and y is independently an integer between 1 and 20; and z is an integer between 5 and 90; and one or more of:
(ii) a phospholipid;
(iii) a steroid;
(iv) a payload; and
(v) an additional lipid component.

159. The particle of embodiment 158, wherein the particle is a liposome, noisome, or lipid nanoparticle.

160. The particle of embodiment 158, wherein the particle is a liposome.

161. The particle of embodiment 158, wherein the particle is a noisome.

162. The particle of embodiment 158, wherein the particle is a lipid nanoparticle.

163. The particle of any one of embodiments 158-162, wherein the payload is selected from a protein, enzyme, peptide, nucleic acid, small molecule, or lipid.

164. The particle of any one of embodiments 158-162, wherein the payload is a protein.

165. The particle of any one of embodiments 158-162, wherein the payload is an enzyme.

166. The particle of any one of embodiments 158-162, wherein the payload is a peptide.

167. The particle of any one of embodiments 158-162, wherein the payload is a nucleic acid.

168. The particle of any one of embodiments 158-162, wherein the payload is a small molecule.

169. The particle of any one of embodiments 158-162, wherein the payload is a lipid.

170. The particle of any one of embodiments 158-162, wherein the payload is an mRNA.

171. A method of treating a disease, disorder, or condition in a subject, the method comprising administering to the subject a particle comprising:
(i) a polymer of Formula (I-d):

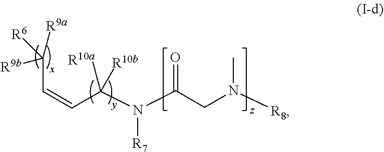

or a pharmaceutically acceptable salt thereof, wherein each of R⁶, R⁷, and R⁸ is independently hydrogen, heteroalkyl, alkyl, alkenyl, or alkynyl; each of R^{9a}, R^{9b}, R^{10a}, and R^{10b} is independently hydrogen, alkyl, or halo; each of x and y is independently an integer between 1 and 20; and z is an integer between 5 and 90;
and one or more of:
(ii) a phospholipid;
(iii) a steroid;
(iv) a payload; and
(v) an additional lipid component.

172. The particle of embodiment 171, wherein the particle is a liposome, noisome, or lipid nanoparticle.

173. The particle of any one of embodiments 171-172, wherein the payload is selected from a protein, enzyme, peptide, nucleic acid, small molecule, or lipid.

174. The particle of any one of embodiments 171-172, wherein the payload is a protein.

175. The particle of any one of embodiments 171-172, wherein the payload is an enzyme.

176. The particle of any one of embodiments 171-172, wherein the payload is a peptide.

177. The particle of any one of embodiments 171-172, wherein the payload is a nucleic acid.

178. The particle of any one of embodiments 171-172, wherein the payload is a small molecule.

179. The particle of any one of embodiments 171-172, wherein the payload is a lipid.

### EXAMPLES

In order that the disclosure described herein may be more fully understood, the following examples are set forth. The examples described in this application are offered to illustrate the particles, compositions and methods provided herein and are not to be construed in any way as limiting their scope.

### Example 1. Synthesis of Lipid Nanoparticles Containing Polysarcosine Lipid Conjugates

Lipid nanoparticles will be prepared by mixing an aqueous phase (0.1-0.3 mg/mL mRNAdiluted in 0.1 M citrate buffer) with an organic phase comprising a mixture of cationic lipid: polysarcosine lipid conjugate:phospholipid:cholesterol dissolved in ethanol at the ratios given in Table 1. The mixture will be dialyzed and then will be concentrated using Centrifugal Filters with a 30 kDa cutoff.

TABLE 1
Organic phase composition of lipid nanoparticle compositions

| | Mole Percentage |
|---|---|
| Organic Phase Composition | of Components |
| Octyl-NH-poly(Sar₃₀):DSPC:Cholesterol:1,2- | 1.5:38.5:10:50 |
| dioleoyloxy-3-(trimethylammonium)propane | |
| Dodecyl-NH-poly(Sar₁₅):DSPC:Cholesterol:1,2- | 1.5:38.5:10:50 |
| dioleoyloxy-3-(trimethylammonium)propane | |
| Decyl-NH-poly(Sar₁₅):DSPC:Cholesterol:1,2- | 1.5:38.5:10:50 |
| dioleoyloxy-3-(trimethylammonium)propane | |
| Octadecyl-NH-poly(Sar₃₀):DSPC:Cholesterol:1,2- | 1.5:38.5:10:50 |
| dioleoyloxy-3-(trimethylammonium)propane | |
| Oleyl-NH-poly(Sar₃₀):DSPC:Cholesterol:1,2- | 1.5:38.5:10:50 |
| dioleoyloxy-3-(trimethylammonium)propane | |
| Oleyl-NH-poly(Sar₁₅):DSPC: Cholesterol:1,2- | 10:38.5:10:40 |
| dioleoyloxy-3-(trimethylammonium)propane | |
| Oleyl-NH-poly(Sar₄₅):DSPC:Cholesterol:1,2- | 15:38.5:10:35 |
| dioleoyloxy-3-(trimethylammonium)propane | |
| (4-hydroxybutyl)azanediyl bis(hexane-6,1- | 46.3:1.6:42.7:9.4 |
| diyl)bis(2-hexyldecanoate) (ALC-0315):oleyl- | |
| NH-poly(Sar₁₅):cholesterol:1,2-distearoyl- | |
| sn-glycero-3-phosphocholine (DSPC) | |
| (4-hydroxybutyl)azanediyl bis(hexane-6,1- | 46.3:1.6:42.7:9.4 |
| diyl)bis(2-hexyldecanoate) (ALC-0315):oleyl- | |
| NH-poly(Sar₂₅):cholesterol:1,2-distearoyl- | |
| sn-glycero-3-phosphocholine (DSPC) | |
| (4-hydroxybutyl)azanediyl bis(hexane-6,1- | 46.3:1.6:42.7:9.4 |
| diyl)bis(2-hexyldecanoate) (ALC-0315):oleyl- | |
| NH-poly(Sar₃₅):cholesterol:1,2-distearoyl- | |
| sn-glycero-3-phosphocholine (DSPC) | |
| (4-hydroxybutyl)azanediyl bis(hexane-6,1- | 46.3:1.6:42.7:9.4 |
| diyl)bis(2-hexyldecanoate) (ALC-0315):oleyl- | |
| NH-poly(Sar₄₅):cholesterol:1,2-distearoyl- | |
| sn-glycero-3-phosphocholine (DSPC) | |
| Heptadecan-9-yl 8-((2-hydroxyethyl)(6-oxo- | 50:1.5:38.5:10 |
| 6-(undecyloxy)hexyl)amino)octanoate (SM- | |
| 102):oleyl-NH-poly(Sar₁₅):cholesterol:1,2- | |
| distearoyl-sn-glycero-3-phosphocholine (DSPC) | |
| Heptadecan-9-yl 8-((2-hydroxyethyl)(6-oxo- | 50:1.5:38.5:10 |
| 6-(undecyloxy)hexyl)amino)octanoate (SM- | |
| 102):oleyl-NH-poly(Sar₂₅):cholesterol:1,2- | |
| distearoyl-sn-glycero-3-phosphocholine (DSPC) | |
| Heptadecan-9-yl 8-((2-hydroxyethyl)(6-oxo- | 50:1.5:38.5:10 |
| 6-(undecyloxy)hexyl)amino)octanoate (SM- | |
| 102):oleyl-NH-poly(Sar₃₅):cholesterol:1,2- | |
| distearoyl-sn-glycero-3-phosphocholine (DSPC) | |
| Heptadecan-9-yl 8-((2-hydroxyethyl)(6-oxo- | 50:1.5:38.5:10 |
| 6-(undecyloxy)hexyl)amino)octanoate (SM- | |
| 102):oleyl-NH-poly(Sar₄₅):cholesterol:1,2- | |
| distearoyl-sn-glycero-3-phosphocholine (DSPC) | |
| N,N-dimethyl-2,3-dioleyloxypropylamine | 40:2.5:47.5:10 |
| (DODMA):oleyl-NH-poly(Sar₁₅):cholesterol:1,2- | |
| distearoyl-sn-glycero-3-phosphocholine (DSPC) | |
| N,N-dimethyl-2,3-dioleyloxypropylamine | 40:2.5:47.5:10 |
| (DODMA):oleyl-NH-poly(Sar₂₅):cholesterol:1,2- | |
| distearoyl-sn-glycero-3-phosphocholine (DSPC) | |
| N,N-dimethyl-2,3-dioleyloxypropylamine | 40:2.5:47.5:10 |
| (DODMA):oleyl-NH-poly(Sar₃₅):cholesterol:1,2- | |
| distearoyl-sn-glycero-3-phosphocholine (DSPC) | |
| N,N-dimethyl-2,3-dioleyloxypropylamine | 40:2.5:47.5:10 |
| (DODMA):oleyl-NH-poly(Sar₄₅):cholesterol:1,2- | |
| distearoyl-sn-glycero-3-phosphocholine (DSPC) | |

### Example 2. Lipid Nanoparticle Formulations Containing Polysarcosine Lipid Conjugates

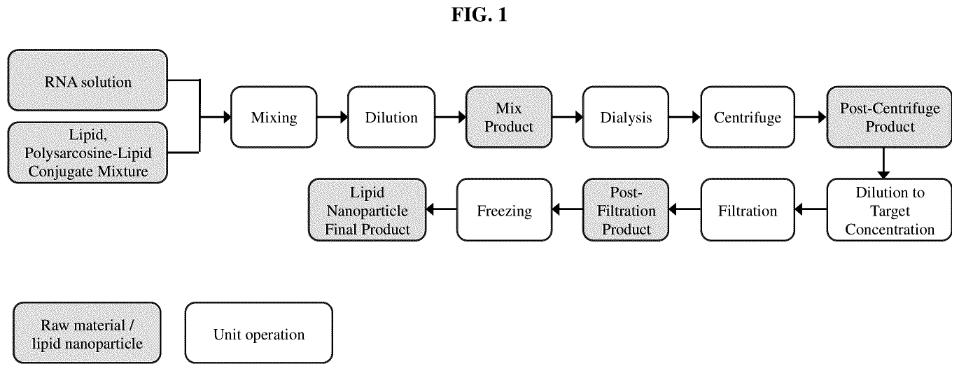

Lipid nanoparticle (LNP) formulations comprising polysarcosine-lipid conjugates enclosing an exemplary cargo (RNA), which is approximately 2 kDa in size, were prepared according to the flow diagram shown in FIG. 1. The mRNA expressing firefly luciferase (Fluc; ˜2k) was obtained from CATUG. SM-102, ALC-0315, ALC-0159 were obtained from SINOPEG. DSPC, cholesterol, and DMG-PEG 2000 (DMG-PEG-2K) were obtained from AVT Pharma. Oleyl-Sar15 was obtained from Curapath. Oleyl-Sar30 and oleyl-Sar10 were manufactured by Calusa Bio.

The mixing instrument used was a NanoAssemblr Ignite obtained from Precision NanoSystems. The volume of the aqueous RNA solution prior to mixing was between 4-11 mL with a density of ˜1 g/mL. The volume of the lipid, polysarcosine-lipid conjugate mixture (in 95:5, volume/volume, ethanol:water) was 1/3 (volume/volume) of the RNA solution. Dialysis (10K MWCO) was performed at 2-8° C. overnight with stirring. Centrifugation concentration (30K MWCO) was performed at 2-8° C., 2000 to 4000×g. Filtration was performed with a 0.22 μm PES filter.

Particle diameters and polydispersity (PDI) were measured with a Wyatt Dynapro Plate Reader III. Encapsulation efficiency (ELE %) was determined via a fluorescence-based assay (RiboGreen obtained from Thermo Fisher) measured on a SpectraMax iD5 microplate reader. mRNA content was quantified via HPLC.

LNP formulations LNP-01 to LNP-04 were prepared and evaluated at the Mix Product step depicted in the flow diagram shown in FIG. 1. The molar percentages of components and analytics are listed in Table 2 and Table 3 shown below.

TABLE 2
Molar percentage of the components comprising
LNP formulations LNP-01 to LNP-04

| | | | | Choles- | DMG- | Oleyl- |
|---|---|---|---|---|---|---|
| LNP # | SM-102 | ALC-0315 | DSPC | terol | PEG2K | pSar15 |
| LNP-01 | 50% | | 10% | 38.5% | 1.5% | — |
| (control) | | | | | | |
| LNP-02 | — | 47.4% | 10% | 40.8% | 1.9% | — |
| (control) | | | | | | |
| LNP-03 | 50% | | 10% | 38.5% | — | 1.5% |
| LNP-04 | — | 47.4% | 10% | 41.2% | — | 1.5% |

TABLE 3
Analytical results for LNP formulations LNP-01
to LNP-04 at the Mixed Product step.

| LNP # | Particle Diameter | | PDI | EE % |
|---|---|---|---|---|
| LNP-01 (control) | 63.1 | nm | 0.14 | 96.1% |
| LNP-02 (control) | 79.9 | nm | 0.19 | 95.6% |
| LNP-03 | 84.8 | nm | 0.25 | 92.6% |
| LNP-04 | 135.5 | nm | 0.15 | 77.5% |

LNP formulations LNP-03, & LNP-05 to LNP-10 were prepared to evaluate, for example, the impact of the length of the polysarcosine chain length of the poly sarcosine-lipid conjugates and evaluated at the Mixed Product step depicted in the flow diagram shown in FIG. 1. The molar percentages of components and analytics are listed in Table 4 and Table 5.

TABLE 4
Molar percentage of the components comprising
LNP formulations LNP-03, & LNP-05 to LNP-10.

| | | | | Oleyl- | Oleyl- | Oleyl- |
|---|---|---|---|---|---|---|
| LNP # | SM-102 | DSPC | Cholesterol | pSar10 | pSar15 | pSar30 |
| LNP-05 | 50% | 10% | 38.5% | 1.5% | — | — |
| LNP-06 | 50% | 10% | 37.5% | 2.5% | — | — |
| LNP-03 | 50% | 10% | 38.5% | — | 1.5% | — |
| LNP-08 | 50% | 10% | 37.5% | — | 2.5% | — |
| LNP-09 | 50% | 10% | 38.5% | — | — | 1.5% |
| LNP-10 | 50% | 10% | 37.5% | — | — | 2.5% |

TABLE 5
Analytical results for LNP formulations LNP-03,
& LNP-05 to LNP-10 at the Mixed Product step.

| | Polysarcosine- | Particle | | |
|---|---|---|---|---|
| LNP # | lipid Conjugate | Diameter | PDI | EE % |
| LNP-05 | Oleyl-pSar10 | 86.3 nm | 0.26 | 90.9% |
| LNP-06 | Oleyl-pSar10 | 87.5 nm | 0.31 | 92.3% |
| LNP-03 | Oleyl-pSar15 | 84.8 nm | 0.25 | 92.6% |
| LNP-08 | Oleyl-pSar15 | 78.9 nm | 0.33 | 95.3% |
| LNP-09 | Oleyl-pSar30 | 79.4 nm | 0.35 | 94.9% |
| LNP-10 | Oleyl-pSar30 | 69.5 nm | 0.34 | 97.6% |

LNP formulations LNP-11 to LNP-22 were prepared to evaluate, for example, the impact of dilution buffer and dilution factor used and were evaluated at the Mixed Product step depicted in the flow diagram shown in FIG. 1. Dilution buffer was either CBS/Tris (citrate buffered saline+3% 1M Tris) or 50 mM Tris. The molar percentages of components and analytics are listed in Table 6 and Table 7.

TABLE 6
Molar percentage of the components comprising LNP formulations,
dilution buffer, and dilution factor LNP-11 to LNP-22.

| | | | | Oleyl- | Oleyl- | Dilution | Dilution |
|---|---|---|---|---|---|---|---|
| LNP # | SM-102 | DSPC | Cholesterol | pSar15 | pSar30 | Buffer | Factor |
| LNP-11 | 50% | 10% | 37.5% | 2.5% | — | CBS/Tris | 2x |
| LNP-12 | 50% | 10% | 37.5% | 2.5% | — | CBS/Tris | 4x |
| LNP-13 | 50% | 10% | 37.5% | 2.5% | — | CBS/Tris | 6x |
| LNP-14 | 50% | 10% | 37.5% | 2.5% | — | 50 mM Tris | 2x |
| LNP-15 | 50% | 10% | 37.5% | 2.5% | — | 50 mM Tris | 4x |
| LNP-16 | 50% | 10% | 37.5% | 2.5% | — | 50 mM Tris | 6x |
| LNP-17 | 50% | 10% | 37.5% | — | 2.5% | CBS/Tris | 2x |
| LNP-18 | 50% | 10% | 37.5% | — | 2.5% | CBS/Tris | 4x |
| LNP-19 | 50% | 10% | 37.5% | — | 2.5% | CBS/Tris | 6x |
| LNP-20 | 50% | 10% | 37.5% | — | 2.5% | 50 mM Tris | 2x |
| LNP-21 | 50% | 10% | 37.5% | — | 2.5% | 50 mM Tris | 4x |
| LNP-22 | 50% | 10% | 37.5% | — | 2.5% | 50 mM Tris | 6x |

TABLE 7
Analytical results for LNP formulations LNP-11
to LNP-22 at the Mixed Product step.

| | Polysarcosine- | Dilution | Dilution | Particle | | |
|---|---|---|---|---|---|---|
| LNP # | lipid Conjugate | Buffer | Factor | Diameter | PDI | EE % |
| LNP-11 | Oleyl-pSar15 | CBS/Tris | 2x | 84.2 nm | 0.27 | 90.7% |
| LNP-12 | Oleyl-pSar15 | CBS/Tris | 4x | 87.7 nm | 0.28 | 93.5% |
| LNP-13 | Oleyl-pSar15 | CBS/Tris | 6x | 81.5 nm | 0.27 | 94.7% |
| LNP-14 | Oleyl-pSar15 | 50 mM Tris | 2x | 131.1 nm | 0.11 | 51.2% |
| LNP-15 | Oleyl-pSar15 | 50 mM Tris | 4x | 98.4 nm | 0.36 | 92.6% |
| LNP-16 | Oleyl-pSar15 | 50 mM Tris | 6x | 95.4 nm | 0.42 | 94.6% |
| LNP-17 | Oleyl-pSar30 | CBS/Tris | 2x | 67.4 nm | 0.18 | 94.7% |
| LNP-18 | Oleyl-pSar30 | CBS/Tris | 4x | 65.8 nm | 0.20 | 96.4% |
| LNP-19 | Oleyl-pSar30 | CBS/Tris | 6x | 65.2 nm | 0.20 | 96.6% |
| LNP-20 | Oleyl-pSar30 | 50 mM Tris | 2x | 78.3 nm | 0.17 | 94.9% |
| LNP-21 | Oleyl-pSar30 | 50 mM Tris | 4x | 78.7 nm | 0.17 | 96.0% |
| LNP-22 | Oleyl-pSar30 | 50 mM Tris | 6x | 73.4 nm | 0.11 | 97.0% |

A selection of LNP formulations LNP-11 to LNP-22 were further processed and evaluated at the Lipid Nanoparticle Final Product step depicted in the flow diagram shown in FIG. 1. The analytics of the formulations are listed in Table 8.

TABLE 8
Analytical results for LNP formulations LNP-11 to 13, LNP-15
to 19, and LNP-21 to 22 at the Final Product step.

| | Polysarcosine- | Dilution | Dilution | Particle | | | | mRNA |
|---|---|---|---|---|---|---|---|---|
| LNP # | lipid Conjugate | Buffer | Factor | Diameter | | PDI | EE % | Content* |
| LNP-11 | Oleyl-pSar15 | CBS/Tris | 2x | 106.6 | nm | 0.20 | 90.7% | 0.09 mg/mL |
| LNP-12 | Oleyl-pSar15 | CBS/Tris | 4x | 119.5 | nm | 0.28 | 93.4% | 0.08 mg/mL |
| LNP-13 | Oleyl-pSar15 | CBS/Tris | 6x | 98.3 | nm | 0.19 | 93.8% | 0.09 mg/mL |
| LNP-15 | Oleyl-pSar15 | 50 mM | 4x | 101.4 | nm | 0.23 | 91.7% | 0.09 |
| | | Tris | | | | | | |
| LNP-16 | Oleyl-pSar15 | 50 mM | 6x | 120.1 | nm | 0.30 | 92.0% | 0.09 |
| | | Tris | | | | | | |
| LNP-17 | Oleyl-pSar30 | CBS/Tris | 2x | 78.9 | nm | 0.15 | 94.7% | 0.10 |
| LNP-18 | Oleyl-pSar30 | CBS/Tris | 4x | 80.5 | nm | 0.14 | 95.3% | 0.10 |
| LNP-19 | Oleyl-pSar30 | CBS/Tris | 6x | 84.0 | nm | 0.16 | 95.6% | 0.10 |
| LNP-21 | Oleyl-pSar30 | 50 mM | 4x | 84.4 | nm | 0.17 | 95.2% | 0.10 |
| | | Tris | | | | | | |
| LNP-22 | Oleyl-pSar30 | 50 mM | 6x | 79.9 | nm | 0.14 | 96.5% | 0.10 |

Tris

*Target mRNA content = 0.1 mg/mL

### EQUIVALENTS AND SCOPE

This application refers to various issued patents, published patent applications, journal articles, and other publications, all of which are incorporated herein by reference in their entirety. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present disclosure, as defined in the following claims.

1. A particle comprising:
(i) a polymer of Formula (I-d): 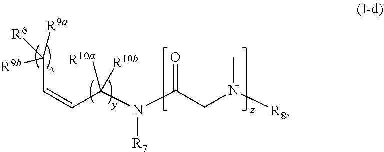
or a pharmaceutically acceptable salt thereof, wherein each of R⁶, R⁷, and R⁸ is independently hydrogen, heteroalkyl, alkyl, alkenyl, or alkynyl; each of R^{9a}, R^{9b}, R^{10a}, and R^{10b} is independently hydrogen, alkyl, or halo; each of x and y is independently an integer between 1 and 20; and z is an integer between 5 and 90;
and one or more of:
(ii) a phospholipid;
(iii) a steroid;
(iv) a payload; and
(v) an additional lipid component.

2. The particle of claim 1, comprising (ii).

3. The particle of claim 1, comprising (iii).

4. The particle of claim 1, comprising (iv).

5. The particle of claim 1, comprising (v).

6. The particle of claim 1, wherein each of R⁶, R⁷, R⁸, R^{9a}, R^{9b}, R^{10a}, and R^{10b} is independently hydrogen.

7. The particle of any one of the preceding claims, wherein z is 5-50.

8. The particle of claim 1, wherein x is between 5-10 (e.g., between 6-8).

9. The particle of claim 1, wherein y is between 5-10 (e.g., between 6-8).

10. The particle of claim 1, wherein the polymer of Formula (I-d) is present at a mole percent of about 1% to 60%.

11. The particle of claim 1, wherein the polymer of Formula (I-d) is present at a mole percent of about 1% to 20%.

12. The particle of claim 1, wherein the polymer of Formula (I-d) is present at a mole percent of about 1% to 15%.

13. The particle of claim 1, wherein the particle is a liposome, noisome, or lipid nanoparticle.

14. The particle of claim 1, wherein the phospholipid is selected from a phosphatidylcholine, phosphatidylethanolamine, phosphatidylserine, and phosphatidylglycerol.

15. The particle of claim 1, wherein the phospholipid is a compound of any one of Formulas (II)-(II-e), e.g., as described herein.

16. The particle of claim 1, wherein the steroid is a compound of any one of Formulas (III)-(III-b), e.g., as described herein.

17. The particle of claim 1, wherein the steroid is cholesterol or a cholesterol derivative.

18. The particle of claim 1, wherein the payload is selected from a protein, enzyme, peptide, nucleic acid, small molecule, or lipid.

19. The particle of claim 18, wherein the payload is a nucleic acid.

20. The particle of claim 19, wherein the nucleic acid is an mRNA molecule.

21. The particle of claim 1, wherein the additional lipid component is a cationic lipid.

22. The particle of claim 1, wherein the additional lipid component is a compound of Formulas (IV)-(IV-b), e.g., as described herein.

23. A lipid nanoparticle comprising:
(i) a polymer of Formula (I-d): 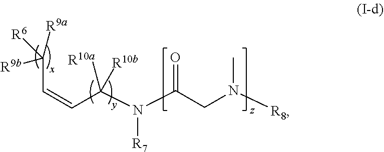
or a pharmaceutically acceptable salt thereof, wherein each of R⁶, R⁷, and R⁸ is independently hydrogen, heteroalkyl, alkyl, alkenyl, or alkynyl; each of R^{9a}, R^{9b}, R^{10a}, and R^{10b} is independently hydrogen, alkyl, or halo; each of x and y is independently an integer between 1 and 20; and z is an integer between 5 and 90;
and one or more of:
(ii) a phospholipid;
(iii) a steroid;
(iv) a payload; and
(v) an additional lipid component.

24. A lipid nanoparticle comprising:
(i) a polymer of Formula (I-d): 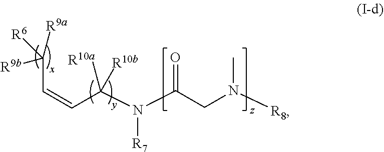
or a pharmaceutically acceptable salt thereof, wherein each of R⁶, R⁷, and R⁸ is independently hydrogen, heteroalkyl, alkyl, alkenyl, or alkynyl; each of R^{9a}, R^{9b}, R^{10a}, and R^{10b} is independently hydrogen, alkyl, or halo; each of x and y is independently an integer between 1 and 20; and z is an integer between 5 and 90:
(ii) a phospholipid;
(iii) a steroid;
(iv) a payload; and
(v) an additional lipid component.

25. A lipid nanoparticle comprising:
(i) a polymer having the structure: 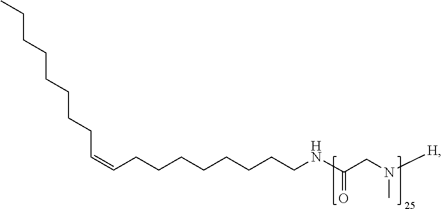
or a pharmaceutically acceptable salt thereof,
and one or more of:
(ii) a phospholipid;
(iii) a steroid;
(iv) a payload; and
(v) an additional lipid component.

26. A lipid nanoparticle comprising:
(i) a polymer having the structure: 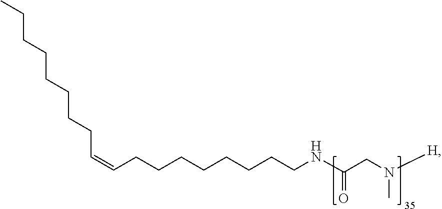
or a pharmaceutically acceptable salt thereof,
(ii) a phospholipid;
(iii) a steroid;
(iv) a payload; and
(v) an additional lipid component.

27. A lipid nanoparticle comprising:
(i) a polymer having the structure: 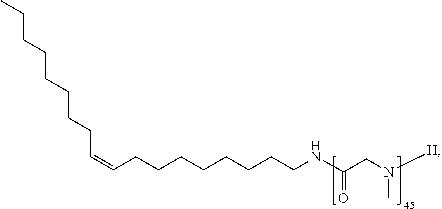
or a pharmaceutically acceptable salt thereof,
(ii) a phospholipid;
(iii) a steroid;
(iv) a payload; and
(v) an additional lipid component.

28. The particle of claim 1, wherein the phospholipid comprises diastearoylphosphatidylcholine (DSPC) or 1,2-dimyristoyl-rac-glycero-3-methoxypolyethylene glycol (DMG-PEG).

29. The particle of claim 28, wherein the DMG-PEG comprises DMG-PEG-2000, DMG-PEG-3000, DMG-PEG-3350, DMG-PEG-4000, DMG-PEG-5000, DMG-PEG-10,000, or DMG-PEG-20,000.

30. The particle of claim 1, wherein the particle comprises a plurality of phospholipids (e.g., DSPC and a DMG-PEG).

31. The particle of any one of claims 28-30, wherein the concentration of DSPC provided in the particle or lipid nanoparticle formulation is between 5-15%.

32. The particle of claim 1, wherein the concentration of DMG-PEG (e.g., DMG-PEG-2,000) provided in the particle or lipid nanoparticle formulation is between 0.5-5%.

33. The particle of claim 1, wherein the polymer of Formula (I-d) is selected from oleyl-pSar10, oleyl-pSar15, and oleyl-pSar30.

34. The particle of claim 1, wherein the concentration of polymer of Formula (I-d) is between 0.5-5%.

35. The particle of claim 1, wherein the diameter of the particle or lipid nanoparticle is between 50 nm-1 mm (e.g., 50 nm-750 nm, 50 nm-500 nm, 50-250 nm).

36. The particle of claim 1, wherein the diameter of the particle or lipid nanoparticle is between 50 nm-200 nm (e.g., 50 nm-150 nm).

37. The particle of claim 1, wherein the polydispersity of the particle or lipid nanoparticle is between 0.05-0.5 (e.g., 0.1-0.4, 0.1-0.3, 0.1-0.2).

38. A method of delivering a payload to a subject or cell, the method comprising administering to the subject or cell a particle comprising:
(i) a polymer of Formula (I-d): 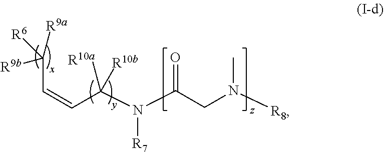
or a pharmaceutically acceptable salt thereof, wherein each of R⁶, R⁷, and R⁸ is independently hydrogen, heteroalkyl, alkyl, alkenyl, or alkynyl; each of R^{9a}, R^{9b}, R^{10a}, and R^{10b} is independently hydrogen, alkyl, or halo; each of x and y is independently an integer between 1 and 20; and z is an integer between 5 and 90;
and one or more of:
(ii) a phospholipid;
(iii) a steroid;
(iv) a payload; and
(v) an additional lipid component.

39. The particle of claim 38, wherein the particle is a liposome, noisome, or lipid nanoparticle.

40. The particle of any one of claims 38-39, wherein the payload is selected from a protein, enzyme, peptide, nucleic acid, small molecule, or lipid.

41. A method of treating a disease, disorder, or condition in a subject, the method comprising administering to the subject a particle comprising:
(i) a polymer of Formula (I-d): 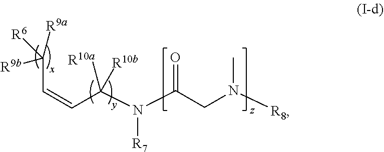
or a pharmaceutically acceptable salt thereof, wherein each of R⁶, R⁷, and R⁸ is independently hydrogen, heteroalkyl, alkyl, alkenyl, or alkynyl; each of R^{9a}, R^{9b}, R^{10a}, and R^{10b} is independently hydrogen, alkyl, or halo; each of x and y is independently an integer between 1 and 20; and z is an integer between 5 and 90;
and one or more of:
(ii) a phospholipid;
(iii) a steroid;
(iv) a payload; and
(v) an additional lipid component.

42. The particle of claim 41, wherein the particle is a liposome, noisome, or lipid nanoparticle.

43. The particle of claim 1, wherein the payload is selected from a protein, enzyme, peptide, nucleic acid, small molecule, or lipid.
